SEQUENCE LISTING (20 sequences)

SEQ ID NO: 1 - DNA, 21 bp, Artificial
  primer
tgtcaccgtggtcaccttctt

SEQ ID NO: 2 - DNA, 23 bp, Artificial
  primer
ccccagatcacgaaggagtctat

SEQ ID NO: 3 - DNA, 21 bp, Artificial
  primer
ttgcactttcgtccctcgccg

SEQ ID NO: 4 - DNA, 18 bp, Artificial
  primer
ccaggaagcccggaagat

SEQ ID NO: 5 - DNA, 19 bp, Artificial
  primer
cggaaggcattggtgaaga

SEQ ID NO: 6 - DNA, 28 bp, Artificial
  primer
tgcccacgtaccgttcctacaatgactc

SEQ ID NO: 7 - DNA, 35 bp, Artificial
  primer
aggaagagagttaaagggggaagaaaatttttaga

SEQ ID NO: 8 - DNA, 56 bp, Artificial
  primer
cagtaatacgactcactatagggagaaggctattacccaatacccaaactaaatcc

SEQ ID NO: 9 - DNA, 32 bp, Artificial
  primer
aggaagagagaagtgttgttgggtgggttttt

SEQ ID NO: 10 - DNA, 54 bp, Artificial
  primer
cagtaatacgactcactatagggagaaggctaacaccttaatccaccccaactt

SEQ ID NO: 11 - DNA, 40 bp, Artificial
  primer
aggaagagagttgtgatggtattatttaaggaatagattt

SEQ ID NO: 12 - DNA, 56 bp, Artificial
  primer
cagtaatacgactcactatagggagaaggctcctcccctccctatataaaaaaaac

SEQ ID NO: 13 - DNA, 31 bp, Artificial
  primer
aggaagagaggttgggggtggttgtaggaat

SEQ ID NO: 14 - DNA, 58 bp, Artificial
  primer
cagtaatacgactcactatagggagaaggctcaactaaccccattacataaacataaa

SEQ ID NO: 15 - DNA, 35 bp, Artificial
  primer
aggaagagagaagttgatttgttaggaagtagggg

SEQ ID NO: 16 - DNA, 56 bp, Artificial
  primer
cagtaatacgactcactatagggagaaggctaacctctctctatacctcaaatccc

SEQ ID NO: 17 - DNA, 25 bp, Artificial
  primer
ggagttttgttttgttttagggttt

SEQ ID NO: 18 - DNA, 25 bp, Artificial
  primer
ctcctattcctccccatataaaaaa

SEQ ID NO: 19 - DNA, 20 bp, Artificial
  primer
tggtgggggatttagaggtt

SEQ ID NO: 20 - DNA, 26 bp, Artificial
  primer
aactacaaaaacaccataaaaaacca

(19) United States
(12) Patent Application Publication
(10) Pub. No.: US 2019/0367986 A1
(43) Pub. Date: Dec. 5, 2019

(54) GENE-SPECIFIC DNA METHYLATION CHANGES PREDICT REMISSION IN ANCA-ASSOCIATED VASCULITIS PATIENTS

(71) Applicant: The University of North Carolina at Chapel Hill, Chapel Hill, NC (US)

(72) Inventors: Britta Jones, Seattle, WA (US); Ronald Falk, Chapel Hill, NC (US); Dominic Ciavatta, Chapel Hill, NC (US)

(21) Appl. No.: 16/341,202

(22) PCT Filed: Oct. 11, 2017
(86) PCT No.: PCT/US2017/056115

§ 371 Date: Apr. 11, 2019

Related U.S. Application Data
(60) Provisional application No. 62/406,919, filed on Oct. 11, 2016.

(51) Int. Cl. C12Q 1/6883

(52) U.S. Cl. CPC C12Q 1/6883; C12Q 2600/106; C12Q 2600/154; C12Q 2600/118

(57) ABSTRACT

The present invention provides compositions and methods for identifying likelihood of relapse/remission in a subject having ANCA-associated vasculitis (AAV).

### PRIORITY STATEMENT

This application is a 35 U.S.C. § 371 national phase application of International Application Serial No. PCT/US2017/056115, filed Oct. 11, 2017, which claims the benefit, under 35 U.S.C. § 119(e), of U.S. Provisional Application No. 62/406,919, filed Oct. 11, 2016, the entire contents of each of which are incorporated by reference herein.

### STATEMENT OF GOVERNMENT SUPPORT

This invention was made with government support under Grant No. DK058335 awarded by the National Institutes of Health (NIH). The government has certain rights in the invention.

### STATEMENT REGARDING ELECTRONIC FILING OF A SEQUENCE LISTING

A Sequence Listing in ASCII text format, submitted under 37 C.F.R. § 1.821, entitled 5470-832_ST25.txt, 3,826 bytes in size, generated on Apr. 9, 2019 and filed via EFS-Web, is provided in lieu of a paper copy. This Sequence Listing is incorporated by reference into the specification for its disclosures.

### FIELD OF THE INVENTION

This invention is directed to methods and compositions to identify likelihood of relapse or remission in a subject having ANCA-associated vasculitis (AAV).

### BACKGROUND

Anti-neutrophil cytoplasmic autoantibodies (ANCAs) interact with autoantigens, primarily myeloperoxidase (MPO) and proteinase 3 (PR3), to induce ANCA-associated vasculitis (AAV), a systemic autoimmune disease characterized by episodes of destructive vascular and extravascular inflammation. Autoantigen expression is aberrantly elevated in 30 patients with AAV, suggesting that a critical factor in AAV is the dysregulation of autoantigens. Thus, both ANCAs and autoantigen expression are important for the development of AAV.

Another prominent feature of AAV is that therapy-induced disease remission may be punctuated by periods of disease relapse. Factors that promote remission or permit disease relapse are unknown; however, because expression of autoantigens is elevated during active disease clues to disease states may lie in understanding mechanisms regulating autoantigen expression. Two well-known epigenetic modifications capable of inducing gene silencing are histone H3 lysine 27 trimethylation (H3K27me3) and DNA methylation. The relationship between H3K27me3, Polycomb Repressive Complex 2 (PRC2), the H3K27 methyltransferase, and DNA methylation is complex, including evidence in stem cells and cancer for combinatorial associations between H3K27me3 and DNA methylation. The dysregulation of MPO and PRTN3 (the gene that encodes PR3) in patients with active AAV has been linked to reduced H3K27me3. Whether DNA methylation regulates MPO and PRTN3 expression has not been investigated in the context of AAV.

The present invention overcomes previous shortcomings in the art by providing methods and compositions for determining the likelihood of relapse or remission in a subject having AAV.

### SUMMARY OF THE INVENTION

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In one embodiment, the present invention provides a method of identifying a subject having anti-neutrophilic cytoplasmic autoantibody (ANCA)-associated vasculitis (AAV) as having an increased likelihood of relapse of (AAV), comprising: a) determining the methylation status of CpG dinucleotides in the promoter of a PRTN3 gene in a first sample obtained from the subject at a first time point; b) determining the methylation status of CpG dinucleotides in the promoter of a PRTN3 gene in a subsequent sample obtained from the subject at a subsequent time point following the first time point; and c) comparing the methylation status of (a) with the methylation status of (b), wherein a decrease in the methylation status of (b) relative to (a) identifies the subject as having an increased likelihood of relapse of AAV. In some embodiments, the method can further comprise initiating and/or enhancing a treatment regimen of the subject.

A further embodiment of this invention is a method of guiding treatment of a subject having AAV, comprising a) determining the methylation status of CpG dinucleotides in the promoter of a PRTN3 gene in a first sample obtained from the subject at a first time point; b) determining the methylation status of CpG dinucleotides in the promoter of a PRTN3 gene in a subsequent sample obtained from the subject at a subsequent time point following the first time point; c) comparing the methylation status of (a) with the methylation status of (b), wherein a decrease in the methylation status of (b) relative to (a) identifies the subject as having an increased likelihood of relapse of AAV and an increase or no change in methylation status of (b) relative to (a) identifies the subject as not having an increased risk of relapse of AAV; and d) initiating and/or enhancing a treatment regimen in a subject identified as having an increased likelihood of relapse of AAV, or not initiating or enhancing a treatment regimen and/or reducing a treatment regimen in a subject identified as not having an increased risk of relapse of AAV.

In an additional embodiment, the present invention provides a method of evaluating response to treatment for AAV in a subject having AAV, comprising: a) determining the methylation status of CpG dinucleotides in the promoter of a PRTN3 gene in a first sample obtained from the subject at a first time point prior to administration of a treatment for AAV; b) administering to the subject the treatment for AAV; c) determining the methylation status of CpG dinucleotides in the promoter of a PRTN3 gene in a subsequent sample obtained from the subject at a subsequent time point following the treatment for AAV; and d) comparing the methylation status of (a) with the methylation status of (b), wherein an increase in the methylation status of (b) relative to (a) identifies the subject as having a positive response to treatment and a decrease or no change in the methylation status of (b) relative to (a) identifies the subject as having no response to treatment or a negative response to treatment.

Further provided herein is a method of reducing the likelihood of relapse in a subject in remission from AAV, comprising: a) determining the methylation status of CpG dinucleotides in the promoter of a PRTN3 gene in a first sample obtained from the subject at a first time point; b) determining the methylation status of CpG dinucleotides in the promoter of a PRTN3 gene in a subsequent sample obtained from the subject at a subsequent time point; c) comparing the methylation status of (a) with the methylation status of (b), wherein a decrease in the methylation status of (b) relative to (a) identifies the subject as having an increased risk of relapse of AAV; and d) initiating and/or enhancing a treatment regimen of the subject.

As an additional embodiment, the present invention provides a kit of reagents for the detection of methylation of one or more CpG dinucleotides in the promoter of a PRTN3 gene.

Other objects and advantages will become apparent upon a review of the following description and figures.

### BRIEF DESCRIPTION OF THE DRAWINGS

FIGS. 1A-E: Relative DNMT1 expression and DNA methylation at PRTN3 and MPO loci comparing active to remission states. (1A) Two-fold decrease in mean DNMT1 expression in active patients compared to healthy controls; mean expression in remitting patients was 1.5-fold higher than active patients. Bars shown are mean and standard deviation; p<0.025 is considered significant after accounting for multiple testing. (1B) Three PRTN3 amplicons covering: the promoter, a CGI and intron 2, a CGI and exon 5. (1C) Two MPO amplicons covering: a CGI and exon 7; a CGI and exon 5-6. Amplicon-wide cross-sectional DNA methylation patterns at the (1D) PRTN3 promoter and (1E) MPO CGI/exon 5-6. Circles are healthy controls; squares are PR3-ANCA patients; triangles are MPO-ANCA patients. Bars shown are median with interquartile range; p<0.0125 is considered significant after accounting for multiple testing.

FIGS. 2A-B: Correlation of DNA methylation and mRNA expression at PRTN3 and MPO. Total leukocytes from healthy controls (circles); MPO-ANCA patients (triangles) and PR3-ANCA patients (squares). Log transformed correlation between DNA methylation at the (2A) PRTN3 promoter and PRTN3 expression (n=187; r=−0.2828) and (2B) DNA methylation at MPO CGI/exon 5-6 and MPO expression (n=186; r=−0.3155).

FIGS. 3A-B: Longitudinal change in DNA methylation from disease activity to remission. Mean and standard deviation shown; p-values are as different from zero, where p<0.025 is significant after accounting for multiple testing. PR3-ANCA patients are squares and MPO-ANCA patients are triangles. Mean DNA methylation change at the (3A) PRTN3 promoter: PR3-ANCA patients 4.03%, MPO-ANCA patients 3.69% and the (3B) MPO CGI/exon 5-6: PR3-ANCA patients 3.06%, MPO-ANCA patients 4.93%.

FIGS. 4A-H: AAV patients stratified by DNA methylation increase or decrease. Paired patients with (4A) increased DNA methylation and (4B) decreased DNA methylation in disease remission at the PRTN3 promoter. (4C) DNA methylation at the PRTN3 promoter compared to expression of PRNT3 in patients with increased methylation in disease remission (r=−0.3390). (4D) DNA methylation at the PRTN3 promoter compared to expression of PRNT3 in the samples with decreased methylation in disease remission (r=−0.08322). Paired patients with (4E) increased methylation and (4F) decreased methylation in disease remission at MPO CGI/exon 5-6. (4G) DNA methylation at MPO CGI/exon 5-6 compared to expression of MPO in patients with increased methylation in disease remission (r=−0.3735). (4H) DNA methylation at MPO CGI/exon 5-6 compared to expression of MPO in patients with decreased methylation in disease remission (r=−0.08508). P-values of <0.05 are considered significant.

FIGS. 5A-E: DNA methylation and probability of relapse. AAV patients stratified by DNA methylation change and followed until next relapse or last clinic follow-up at the (5A) PRTN3 promoter and (5B) MPO CGI/exon 5-6. AAV patients stratified by DNA methylation change and serotype and followed until next relapse or last clinic follow-up at the (5C) PRTN3 promoter and (5D) MPO CGI/exon 5-6; MPO-ANCA patients with decreased DNA methylation in solid line, MPO-ANCA patients with increased DNA methylation in dashed line, PR3-ANCA patients with decreased DNA methylation in solid line, PR3-ANCA patients with increased DNA methylation in dashed line. (5E) AAV patients stratified by serotype and followed until next relapse or last clinic follow-up at the PRTN3 promoter. Numbers at bottom of graphs correspond to the number of patients in each group who have not relapsed and have been followed up in clinic. P-values <0.05 are considered significant.

FIGS. 6A-G: Epigenome-wide DNA methylation in patients with AAV. Genome-wide DNA methylation 6(A) beta-value (methylated signal/(methylated signal+unmethylated signal)) and (6B) Mean M value (log(methylated signal/unmethylated signal)) at 485,512 CpGs for 4 healthy controls, 4 MPO-ANCA active patients and 6 PR3-ANCA active patients. Gene-specific median methylation at all CpG dinucleotides in healthy controls (left) compared to AAV patients (right) at (6C) PRTN3, (6D) MPO, (6E) ELANE, (6F) LTF and (6G) BPI. For each graph, the line represents the median, the box the first and third quartiles, and the whiskers represent the maximum and minimum values or 1.5 interquartile range.

FIGS. 7A-D: DNA methylation at additional loci within PRTN3 and MPO. Cross-sectional methylation at (7A) PRTN3 CGI/exon 5 and (7B) MPO CGI/exon 7. Healthy controls (circles); PR3-ANCA patients (squares); MPO-ANCA patients (triangles); bars shown are median with interquartile range; p<0.0125 is considered significant after accounting for multiple testing. Mean longitudinal methylation change from disease activity to remission at (7C) PRTN3 CGI/exon 5: PR3-ANCA patients 1.49%, MPO-ANCA patients 4.36%; and (7D) MPO CGI/exon 7: PR3-ANCA patients 1.22%, MPO-ANCA patients 9.31%. Error bars are standard deviation; p-values shown are as different from zero, where p<0.025 is significant after accounting for multiple testing.

FIG. 8: Role of glucocorticoid therapy in DNA methylation. DNA methylation at CpG 7, 8 of the PRTN3 promoter for all samples collected from active patients not prescribed glucocorticoid therapy (GC) (circles); active patients prescribed GC for less than or equal to three months (squares) and patients prescribed GC for more than three months (triangles). Bars shown are median DNA methylation; p<0.05 is considered significant.

FIGS. 9A-F: DNA methylation at loci within additional granulocyte genes. Green circles are healthy controls; squares are PR3-ANCA patients; triangles are MPO-ANCA patients. (9A) Cross-sectional and (9B) longitudinal methylation at CGI/intron 2 in PRTN3. (9C) Cross-sectional and (9D) longitudinal methylation at a CGI/exon 1 in lactotransferrin (LTF). (9E) Cross-sectional and (9F) longitudinal methylation at a CGI and exon 2 in elastase (ELANE). Bars are median methylation or median longitudinal methylation change from disease activity to remission.

FIGS. 10A-B: DNA methylation platform comparison. Bisulfite sequencing compared to MassARRAY. Cross-sectional methylation at CpG 1 PRTN3 promoter for (10A) bisulfite sequencing and (10B) MassARRAY. Circles are healthy controls; squares are PR3-ANCA patients; triangles are MPO-ANCA patients. Bars are median and interquartile range for all three graphs; p<0.0125 is considered significant, accounting for multiple testing.

FIGS. 11A-B: Correlation of DNA methylation and mRNA expression at DNMT1. Healthy controls (circles); MPO-ANCA patients (triangles) and PR3-ANCA patients (squares). Log transformed correlation between mnethylation at the (11A) PRTN3 promoter and DNMT1 expression (n=179; r=0.4858) and (11B) correlation between methylation at MPO CGI/exon 5-6 and DNMT1 expression (n=177; r=0.5464).

FIGS. 12A-D: DNA methylation changes at individual CpGs within the PRTN3 promoter and MPO CGI/exons 5-6. Circles are healthy controls; squares are PR3-ANCA patients; triangles are MPO-ANCA patients. Bars shown are median with interquartile range; p<0.0125 is significant after accounting for multiple testing. Cross-sectional methylation at (12A) CpG 7, 8 PRTN3 promoter and (12B) CpG 38 MPO CGI/exons 5-6. Mean longitudinal methylation change from disease activity to remission at (12C) CpG 7, 8 PRTN3 promoter: PR3-ANCA patients 3.85%, MPO-ANCA patients 5.57% and (12D) CpG 38 MPO CGI/exons 5-6: PR3-ANCA patients 2.11%, MPO-ANCA patients 5.95%. For longitudinal graphs, p-values shown are as different from zero where p<0.025 is significant after accounting for multiple testing, bars are mean and standard deviation.

FIGS. 13A-B: Amplicon locations within ELANE and LTF. (13A) Amplicon shown covering part of a CGI and exon 2 within the ELANE. (13B) Amplicon shown covering a CGI and exon 1 within the LTF.

### DETAILED DESCRIPTION OF THE INVENTION

The present invention will now be described more fully hereinafter with reference to the accompanying drawings and specification, in which preferred embodiments of the invention are shown. This invention may, however, be embodied in different forms and should not be construed as limited to the embodiments set forth herein.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a non-viral vector) and the like, is meant to encompass variations of ±20%, ±10%, ±5%, ±1%, ±0.5%, or even ±0.1% of the specified amount.

As used herein, the transitional phrase “consisting essentially of” (and grammatical variants) means that the scope of a claim is to be interpreted to encompass the specified materials or steps recited in the claim, “and those that do not materially affect the basic and novel characteristic(s)” of the claimed invention. Thus, the term “consisting essentially of” when used in a claim of this invention is not intended to be interpreted to be equivalent to “comprising.”

The present invention is based on the unexpected discovery of the utility of DNA methylation patterns of the promoter of the PRTN3 gene as a biomarker for determining the likelihood of relapse or remission in a subject having AAV. Thus, in one embodiment, the present invention provides a method of identifying a subject having anti-neutrophilic cytoplasmic autoantibody (ANCA)-associated vasculitis (AAV) as having an increased likelihood of relapse of (AAV), comprising: a) determining the methylation status of CpG dinucleotides in the promoter of a PRTN3 gene in a first sample obtained from the subject at a first time point; b) determining the methylation status of CpG dinucleotides in the promoter of a PRTN3 gene in a subsequent sample obtained from the subject at a subsequent time point following the first time point; and c) comparing the methylation status of (a) with the methylation status of (b), wherein a decrease in the methylation status of (b) relative to (a) identifies the subject as having an increased likelihood of relapse of AAV.

In some embodiments, the method can further comprising initiating and/or enhancing a treatment regimen of the subject. Nonlimiting examples of a treatment regimen include immunosuppression, DNA methylation, reduction of and/or elimination of and/or avoidance of therapy that decreases DNA methylation, and any combination thereof.

In one embodiment, the present invention provides a method of identifying a subject having AAV as having remission of AAV or an increased likelihood of remission of AAV, comprising: a) determining the methylation status of CpG dinucleotides in the promoter of a PRTN3 gene in a first sample obtained from the subject at a first time point; b) determining the methylation status of CpG dinucleotides in the promoter of a PRTN3 gene in a subsequent sample obtained from the subject at a subsequent time point following the first time point; and c) comparing the methylation status of (a) with the methylation status of (b), wherein an increase in the methylation status of (b) relative to (a) identifies the subject as having an increased likelihood of remission of AAV.

A subsequent time point can be any time point after a first time point, e.g., a second time point, a third time point, a fourth time point, a fifth time point, a sixth time point, a seventh time point, an eighth time point, a ninth time point, a tenth time point, etc. Furthermore the comparison of methylation status can be between any time points. For example, the comparison can be between a second point and fourth time point, a first and a fifth time point, etc., as would be well understood by one of ordinary skill in the art.

In the methods of this invention, the first time point can be during an active disease status of the subject or during a remission status of the subject and any subsequent time points can be during an active disease status of the subject or during a remission status of the subject, in any combination. Thus, for example, the first time point and the subsequent time point can be during active disease status of the subject, the first time point and the subsequent time point can be during remission status of the subject, the first time point can be during active disease status of the subject and the subsequent time point can be during remission status of the subject, or the first time point can be during remission status of the subject and the subsequent time point can be during active disease status of the subject.

In one embodiment, the present invention provides a method of guiding treatment of a subject having AAV, comprising a) determining the methylation status of CpG dinucleotides in the promoter of a PRTN3 gene in a first sample obtained from the subject at a first time point; b) determining the methylation status of CpG dinucleotides in the promoter of a PRTN3 gene in a subsequent sample obtained from the subject at a subsequent time point following the first time point; c) comparing the methylation status of (a) with the methylation status of (b), wherein a decrease in the methylation status of (b) relative to (a) identifies the subject as having an increased likelihood of relapse of AAV and an increase or no change in methylation status of (b) relative to (a) identifies the subject as not having an increased risk of relapse of AAV; and d) initiating and/or enhancing a treatment regimen in a subject identified as having an increased likelihood of relapse of AAV, or not initiating or enhancing a treatment regimen and/or reducing a treatment regimen in a subject identified as not having an increased risk of relapse of AAV.

Further provided herein is a method of evaluating response to treatment for AAV in a subject having AAV, comprising: a) determining the methylation status of CpG dinucleotides in the promoter of a PRTN3 gene in a first sample obtained from the subject at a first time point prior to administration of a treatment for AAV; b) administering to the subject the treatment for AAV; c) determining the methylation status of CpG dinucleotides in the promoter of a PRTN3 gene in a subsequent sample obtained from the subject at a subsequent time point following the treatment for AAV; and d) comparing the methylation status of (a) with the methylation status of (b), wherein an increase in the methylation status of (b) relative to (a) identifies the subject as having a positive response to treatment and a decrease or no change in the methylation status of (b) relative to (a) identifies the subject as having no response to treatment or a negative response to treatment.

Nonlimiting examples of a treatment for AAV include administration of an agent that increases DNA methylation, reduction of, elimination of and/or avoidance of treatment that decreases DNA methylation, an immunosuppressive agent, and any combination thereof.

Nonlimiting examples of an immunosuppressive agent of this invention include mycophenolate mofetil, cyclosporine, azathioprine, plasmapheresis, corticosteroid, oral or intravenous cyclophosphamide, methylprednisolone, methotrexate, rituximab, any other immunosuppressive agent, and any combination thereof.

Nonlimiting examples of an agent that inhibits DNA methylation include 5-azacitidine and decitabine, which are used to inhibit tumors by reactivating tumor suppressors silenced by DNA methylation.

Nonlimiting examples of an agent that increases DNA methylation include platinum based chemotherapy and an agent that increases folate/folic acid metabolism.

Nonlimiting examples of an agent that modulates DNA methylation (either by increasing or decreasing DNA methylation) include an epigenetic modifier such as acbromodomain containing protein, histone acetyl transferase, histone deacetylase, protein methyltransferase, histone methyltransferase, and any combination thereof.

An additional embodiment of this invention is a method of reducing the likelihood of relapse in a subject in remission from AAV, comprising: a) determining the methylation status of CpG dinucleotides in the promoter of a PRTN3 gene in a first sample obtained from the subject at a first time point; b) determining the methylation status of CpG dinucleotides in the promoter of a PRTN3 gene in a subsequent sample obtained from the subject at a subsequent time point; c) comparing the methylation status of (a) with the methylation status of (b), wherein a decrease in the methylation status of (b) relative to (a) identifies the subject as having an increased risk of relapse of AAV; and d) initiating and/or enhancing a treatment regimen of the subject. In some embodiments, the treatment regimen is immunosuppression, DNA methylation, reduction of, elimination of and/or avoidance of therapy that decreases DNA methylation, and any combination thereof.

It is to be understood that the methods of this invention can be employed to reduce the dosage and/or duration of a treatment regimen and/or reduce the side effects of a treatment regimen by monitoring changes in the methylation status of the promoter of the PRTN3 gene of a subject having AAV, wherein a decrease in methylation indicates that the subject is moving towards relapse or is in relapse and an increase or no change in methylation indicates that the subject is moving towards remission, remaining in remission or entering remission.

For example, a subject determined to be in remission of AAV can be monitored over time for changes in methylation status of the promoter of the PRTN3 gene. If a decrease is detected, the subject can initiate a treatment regimen and/or enhance a treatment regimen that the subject may already be on. If an increase or no change is detected over time, the subject can reduce the dosage and/or duration of a treatment regimen or discontinue a treatment regimen. The methylation status of the promoter of the PRTN3 gene of the subject can be monitored at any time interval over any period of days, weeks, months, years, etc., to guide treatment and/or maintenance strategies.

Additional embodiments of this invention include a method of treating a subject having AAV for relapse of AAV, wherein the subject is identified as having a decrease in methylation of the PRTN3 gene of the subject as compared with methylation of the PRTN3 gene of the subject at an earlier time point.

CpG islands are genomic regions that contain a high frequency of CG dinucleotides. Thus, these regions generally have a GC percentage that is greater than about 50% and with an observed/expected CpG ratio that is greater than about 60%. (Gardiner-Garden et al. “CpG islands in vertebrate genomes,” J Mol Biol 196: 261-282 (1987)).

As used herein, the term “methylation” refers to the presence of epigenetic methylation of cytosine residues in DNA at sites where it is not typically present in normal cells.

The PRTN3 gene encodes proteinase 3 (PR3). PR3 is a serine protease enzyme expressed mainly in neutrophil granulocytes. Its exact role in the function of the neutrophil is unknown, but, in human neutrophils, PR3 contributes to the proteolytic generation of antimicrobial peptides. It is also the target of anti-neutrophil cytoplasmic antibodies (ANCAs) of the c-ANCA (cytoplasmic subtype) class, a type of antibody frequently found in the disease granulomatosis with polyangiitis (formerly known as “Wegener's granulomatosis”).

Also as used herein, the terms “promoter” and “promoter element” refer to DNA sequences that regulate the expression of a gene; these sequences can be upstream or downstream or at any location relative to the transcriptional initiation site of the gene, from which they provide their regulatory effect.

A further embodiment of the present invention is a screening assay for identifying an agent that has the effect of increasing DNA methylation of the promoter of the PRTN3 gene. Such an agent may be used as a therapeutic in the treatment and/or management of AAV. Such a screening assay would involve the steps of determining the DNA methylation status of the promoter of the PRTN3 gene in a nucleic acid sample comprising the PRTN3 gene and promoter, e.g., from cells grown in culture or isolated from an in vivo source (i.e., peripheral blood cells). Then the cells containing the nucleic acid with the PRTN3 gene and promoter are contacted with the test agent. Then the nucleic acid is isolated from the cells and analyzed for the DNA methylation status of the promoter of the PRTN3 gene and if there is an increase in DNA methylation status following contact of the agent with the nucleic acid (e.g., in the cells), then the agent is identified as having the effect of increasing DNA methylation of the promoter of the PRTN3 gene.

Detection of methylation of CpG dinucleotides can be carried out according to methods of this invention, as well as any art-known method, including, but not limited to, e.g., methylation specific-polymerase chain reaction (MS-PCR), a method of nucleic acid amplification that is well known in the art. In this assay, bisulfite modification of the DNA sequence allows the detection of differences between methylated and unmethylated alleles. Reaction of the DNA with sodium bisulfite converts all unmethylated cytosines to uracil, which is recognized as thymine by Taq polymerase, but does not affect methylated cytosines. Amplification with primers specific for methylated or unmethylated DNA discriminates between methylated and unmethylated DNA. This assay provides a simple and fast way of surveying multiple samples to detect methylation of cytosines in the region of interest (Widschwendter et al., “Methylation and silencing of the retinoic acid receptor-beta2 gene in breast cancer” J. Natl. Cancer Inst. 92(10):826-832 (2000)).

Other methods known in the art for detection and/or quantitative analysis of DNA methylation include, but are not limited to, a chromatin immunoprecipitation assay (ChIP) (Mulero-Navarro et al., Carcinogenesis 27:1099-1104 (2006); Nakagawachi et al., Oncogene 22:8835-8844 (2003)); MethyLight®, a bisulfite modification-dependent fluorescence-based real time PCR assay (Eads et al. Nucleic Acids Res. 28(8) e32 (2000); Erhlich et al., Oncogene 21:6694-6702 (2002)); pyrosequencing (Lee et al. Clinical Cancer Research 14:2664-2672 (2008); Dejeux et al., J. Mol. Diagn. 9:510-520 (2007)); and the Sequenom® MassARRAY® system (Sequenom, Inc., San Diego, Calif.), which utilizes MALDI-TOF mass spectrometry in combination with RNA base specific cleavage (MassCLEAVE™ kit) (Sequenom, Inc., San Diego, Calif.).

In some embodiments, the present invention provides a kit of reagents for the detection of methylation of one or more CpG dinucleotides in the promoter of a PRTN3 gene.

In other embodiments, the kit also comprises reagents and primers (and optionally probes) for amplification and analysis of CpG-containing nucleic acid of the promoter of the PRTN3 gene.

As used herein, “nucleic acids” encompass both RNA and DNA, including cDNA, genomic DNA, mRNA, synthetic (e.g., chemically synthesized) DNA and chimeras of RNA and DNA. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

An “isolated nucleic acid” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter/promoter element) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence.

The term “isolated” can refer to a nucleic acid or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state.

An isolated cell refers to a cell that is separated from other cells and/or tissue components with which it is normally associated in its natural state. For example, an isolated cell is a cell that is part of a cell culture. An isolated cell can also be a cell that is administered to or introduced into a subject, e.g., to impart a therapeutic or otherwise beneficial effect.

The term “oligonucleotide” refers to a nucleic acid sequence of at least about four nucleotides to about 100 nucleotides, for example, about 15 to 30 nucleotides, or about 20 to 25 nucleotides, which can be used, for example, as a primer in an amplification reaction (e.g., PCR) and/or as a probe in a hybridization assay and/or in a microarray. Oligonucleotides can be natural or synthetic, e.g., DNA, RNA, modified backbones, etc., as are well known in the art. Peptide nucleic acids (PNAs) can also be used as oligonucleotides (e.g., as probes) in the methods of this invention.

A subject of this invention is any animal that is susceptible to AAV as described herein. Examples of subjects of this invention can include, but are not limited to, humans, mammals, non-human primates, dogs, cats, horses, cows, goats, guinea pigs, mice, rats and rabbits, as well as any other domestic or commercially valuable animal including animal models of breast cancer. The subject of this invention can be either gender. The subject can be of any ethnicity (e.g., Caucasian, African-American, African, European-American, white, black, Hispanic, Asian, etc.). A subject of the present invention can further be a subject of this invention that has been previously identified as being at high risk of AAV relapse. Alternatively, a subject of the present invention can be a subject that has not been previously identified as being at high risk of AAV relapse.

A “subject in need thereof” is a subject known to have, or suspected of having, diagnosed with, or at risk of having disease or disorder described herein. A subject of this invention can also include a subject not previously known or suspected to have a disease or disorder as described herein or in need of treatment for disease or disorder as described herein. For example, a subject of this invention can be administered the agents and treatments of this invention even if it is not known or suspected that the subject has a disease or disorder as described herein (e.g., prophylactically). A subject of this invention is also a subject known or believed to be at risk of developing a disease or disorder as described herein.

By the terms “treat,” “treating” or “treatment of” (or grammatically equivalent terms) it is meant that the severity of the subject's disease or disorder is reduced or at least partially improved or ameliorated and/or that some alleviation, mitigation or decrease in at least one clinical symptom is achieved and/or there is a delay in the progression of the disease or disorder, as would be well known in the art. Thus, in some embodiments, the terms “treat,” “treating” or “treatment of,” refer only to therapeutic regimens. In other embodiments, the terms “treat,” “treating” or “treatment of” (or grammatically equivalent terms) refer to both prophylactic and therapeutic regimens.

The terms “prevent,” “preventing” and “prevention” (and grammatical variations thereof) refer to avoidance, prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is delayed and/or is less than what would occur in the absence of the method of the present invention.

An “effective amount,” as used herein, refers to an amount that imparts a desired effect, which is optionally a therapeutic or prophylactic effect.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

Pharmaceutical compositions comprising an agent or composition of this invention and a pharmaceutically acceptable carrier are also provided. The compositions described herein can be formulated for administration in a pharmaceutical carrier in accordance with known techniques. See, e.g., Remington, The Science And Practice of Pharmacy (latest edition). In the manufacture of a pharmaceutical composition according to embodiments of the present invention, the composition of this invention is typically admixed with, inter alia, a pharmaceutically acceptable carrier.

By “pharmaceutically acceptable carrier” is meant a carrier that is compatible with other ingredients in the pharmaceutical composition and that is not harmful or deleterious to the subject. The carrier may be a solid or a liquid, or both, and is preferably formulated with the composition of this invention as a unit-dose formulation, for example, a tablet, which may contain from about 0.01 or 0.5% to about 95% or 99% by weight of the composition. The pharmaceutical compositions are prepared by any of the well-known techniques of pharmacy including, but not limited to, admixing the components, optionally including one or more accessory ingredients. In certain embodiments, the pharmaceutically acceptable carrier is sterile and would be deemed suitable for administration into human subjects according to regulatory guidelines for pharmaceutical compositions comprising the carrier.

Furthermore, a “pharmaceutically acceptable” component such as a salt, carrier, excipient or diluent of a composition according to the present invention is a component that (i) is compatible with the other ingredients of the composition in that it can be combined with the compositions of the present invention without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include any of the standard pharmaceutical carriers such as phosphate buffered saline solutions, water, emulsions such as oil/water emulsion, microemulsions and various types of wetting agents.

Exemplary modes of administration of the compositions of this invention can include oral, rectal, intranodal, transmucosal, topical, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intraperitoneal, intradermal, intrapleural, intracerebral, intracranial, and intraarticular), topical (e.g., to both skin and mucosal surfaces, including airway surfaces, and transdermal administration, and the like, as well as direct tissue or organ injection (e.g., to liver, skeletal muscle, cardiac muscle, diaphragm muscle or brain). The most suitable route in any given case will depend on the nature and severity of the condition being treated and on the nature of the particular protein, peptide, fragment, nucleic acid and/or vector that is being used.

The compositions of the present invention may be administered to a subject in need of treatment prior to, during and/or after onset of the disease or disorder. Thus, the compositions of the present invention can be used to treat ongoing diseases or disorders and/or to prevent diseases or delay the development of diseases or disorders.

In some embodiments, an effective dose or effective amount can comprise one or more (e.g., two or three or four or more) doses of the composition of this invention at any time interval (e.g., hourly, daily, weekly, monthly, yearly, as needed) so as to achieve and/or maintain the desired therapeutic benefit.

The present subject matter will be now be described more fully hereinafter with reference to the accompanying examples, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### EXAMPLES

The following examples provide illustrative embodiments. Certain aspects of the following examples are disclosed in terms of techniques and procedures found or contemplated by the present inventors to work well in the practice of the embodiments. In light of the present disclosure and the general level of skill in the art, those of skill will appreciate that the following examples are intended to be exemplary only and that numerous changes, modifications, and alterations can be employed without departing from the scope of the presently claimed subject matter.

### Example 1

DNA methylation was measured at AAV-related autoantigen genes, MPO and PRTN3, in patients with AAV. A longitudinal analysis showed that DNA methylation at MPO and PRTN3 was (i) reduced in patients with active disease and increased during disease remission and (ii) associated with mRNA expression of these genes. The dynamics of DNA methylation at the PRTN3 promoter revealed AAV patients with increased DNA methylation during disease remission had an increased probability of stable remission. Patients with decreased DNA methylation at the PRTN3 promoter were 4.55 times more likely to relapse; this suggests changes in DNA methylation at the PRTN3 promoter can predict long-term prognosis for AAV patients.

Decreased DNMT1 Expression in AAV Patients Occurs without Global Hypomethylation.

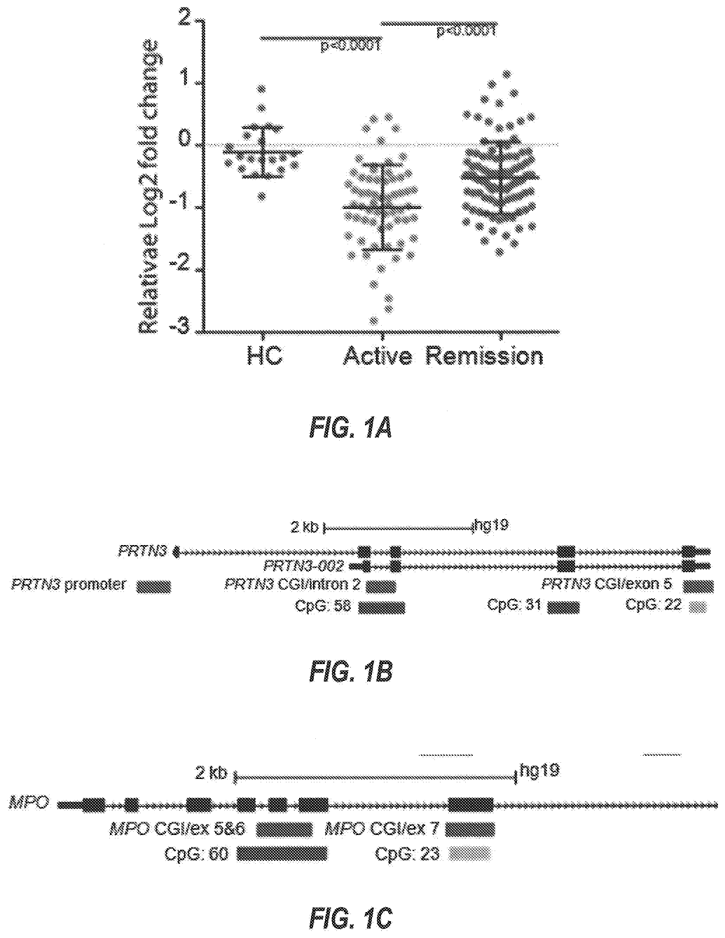

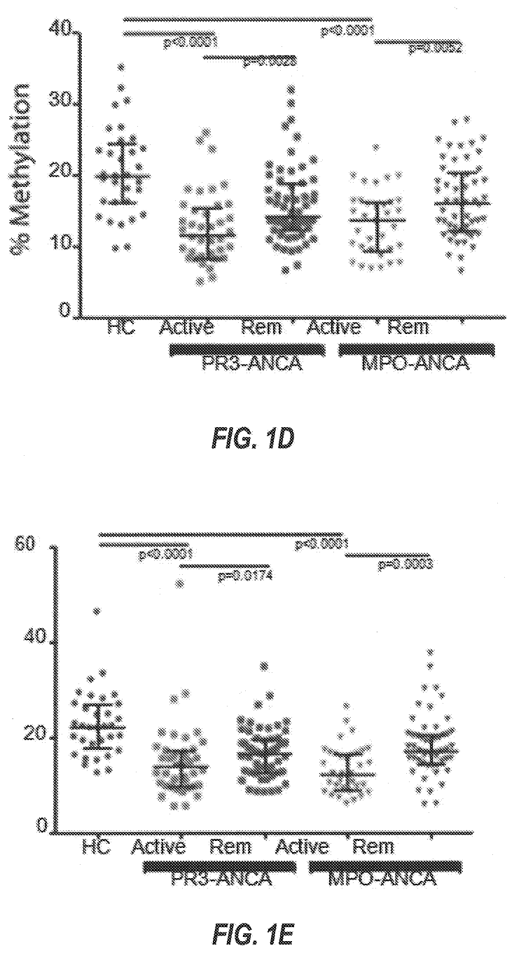

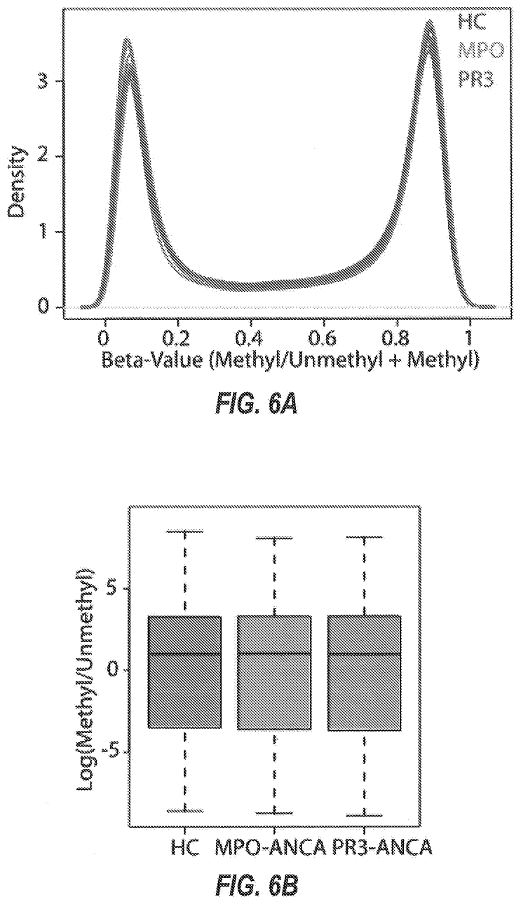

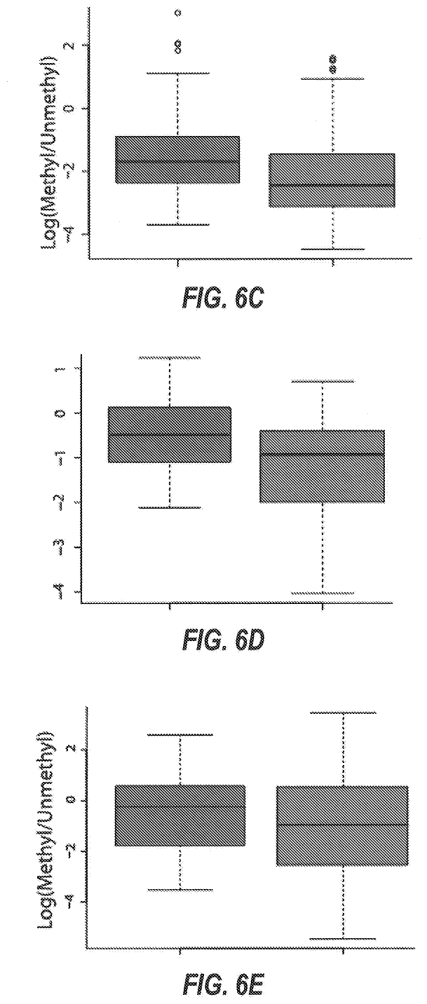

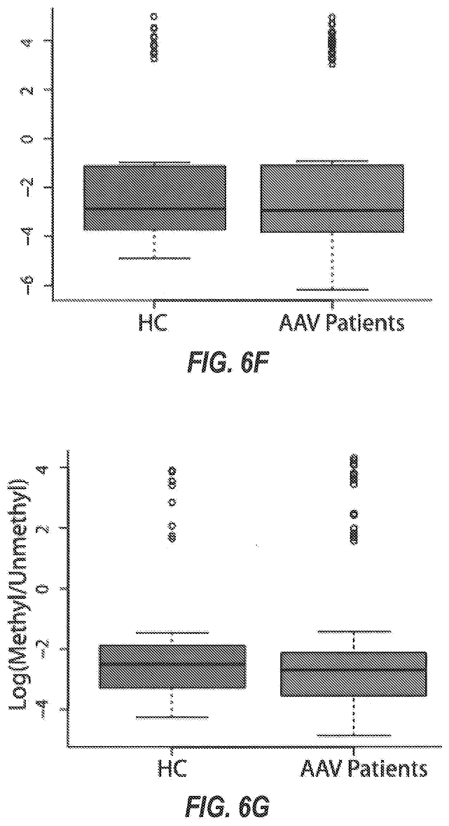

Our investigation of DNA methylation in patients with AAV was sparked by gene expression studies demonstrating that expression of the DNA methyltransferase 1 gene (DNMT1) was decreased in patients with AAV compared to healthy individuals (Yang, et al., Clinical Epigenetics, in press). We confirmed differential DNMT1 expression by quantitative real-time PCR in leukocytes collected from a cohort of AAV patients during disease activity and remission (Table 1). The relative mean DNMT1 expression among AAV patients during disease activity was two-fold less compared to healthy individuals and 1.5-fold higher among patients in disease remission compared to patients with active disease (p<0.0001) (FIG. 1A). To determine if reduced DNMT1 expression in AAV patients with active disease resulted in genome-wide hypomethylation we measured genome-wide DNA methylation. The median DNA methylation among patients with active disease and healthy individuals was nearly identical, indicating no obvious differences in genome-wide DNA methylation despite differences in DNMT1 expression (FIGS. 6A and 6B).

Differential DNA Methylation Restricted to Autoantigen Genes.

Although active patients and healthy controls had similar genome-wide DNA methylation, the median DNA methylation at all CpG dinucleotides in MPO and PRTN3 was less in AAV patients with active disease than healthy controls (FIGS. 6C and 6D). To determine if the differential methylation at MPO and PRTN3 was a shared feature of all genes, we compared the decrease in DNA methylation between active patients and healthy controls within MPO and PRTN3 to the decrease in DNA methylation between active patients and healthy controls at CpGs of all other genes. Of 19,654 unique genes, a greater decrease in DNA methylation in active samples was found for only 881 genes and 953 genes compared to MPO and PRTN3, respectively (Table 2).

To test if differential DNA methylation was a feature of other neutrophil granule genes, methylation was measured at CpG islands (CGIs) in neutrophil elastase (ELANE), lactotransferrin (LTF) and bactericidal/permeability-increasing protein (BPI). We found smaller differences in DNA methylation between AAV patients and healthy controls at ELANE than those found at MPO and PRTN3 (FIG. 6E), resulting in 1591 genes with greater differences in DNA methylation than the difference at ELANE. Our genome-wide DNA methylation studies found no difference between patients and healthy controls at BPI or LTF (FIGS. 6F and 6G). We conclude that differential DNA methylation in AAV patients is restricted to specific loci, including MPO and PRTN3. A potential explanation for the locus specific DNA methylation is targeted DNA methyltransferase activity via complexes of DNMT1 with sequence-specific transcription factors.

AAV Patient-Derived Leukocytes Exhibit Hypomethylation of Loci within MPO and PRTN3.

We screened our larger cohort of paired samples from patients during disease activity and remission for differential DNA methylation at MPO and PRTN3. Three loci in PRTN3 and two loci in MPO were analyzed (FIGS. 1B and 1C). The loci in PRTN3 included the promoter, spanning 15 CpGs, and two CGIs: one contained within exon 2 and the alternative promoter, which we reported was active in AAV patients with active disease, the second is contained within exon 5 and the 3′UTR, where DNA methylation can regulate transcriptional activity. In MPO, a CGI spanning exons 5 and 6 was chosen based on our previous report of DNA methylation at this region. The second region screened in MPO contains a CGI that spans exon 7. Both regions are DNase I sensitive and enriched in transcription factor binding sites, suggesting transcriptional regulatory properties. The MPO promoter was not probed because the promoter is CpG-poor with only seven CpGs within 500 bp of the transcription start site.

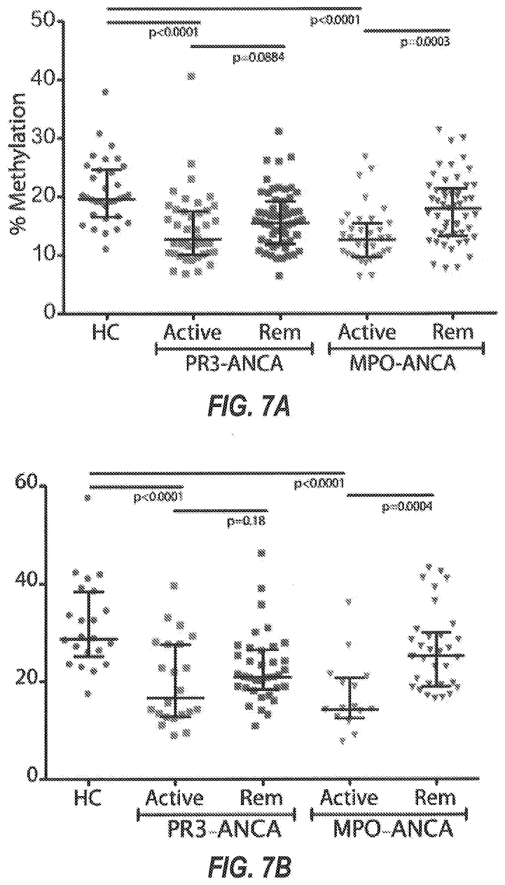

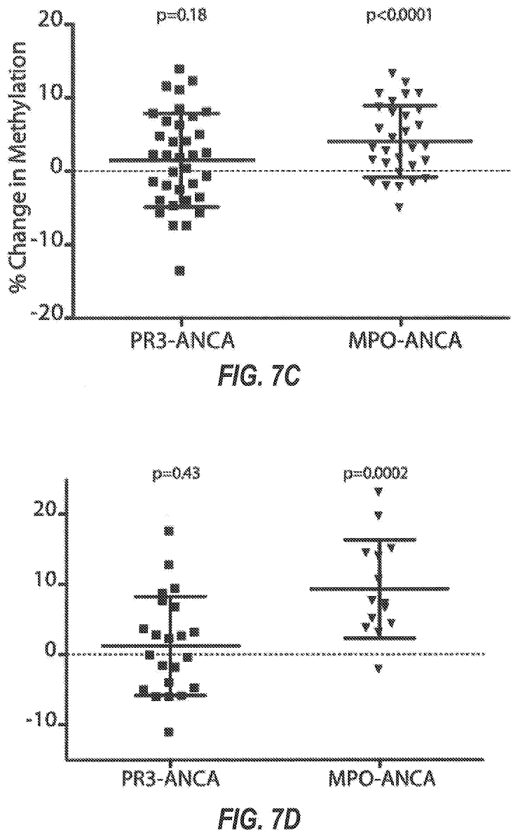

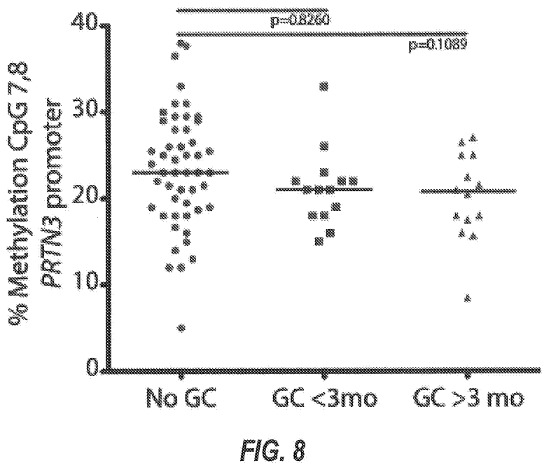

Consistent with the differential methylation at MPO and PRTN3 seen in the genome-wide DNA methylation study, we found that MPO- and PR3-ANCA patients with active disease were hypomethylated compared to healthy individuals at four loci within MPO and PRTN3 (p<0.0001) (FIGS. 1D and 1E; FIGS. 7A and 7B). DNA methylation at these sites rebounded in samples during disease remission. The role of prednisone, a commonly prescribed glucocorticoid therapy (GC), on DNA methylation was examined. DNA methylation of active samples on and off GC show a comparable median methylation at the PRTN3 promoter regardless of time on therapy (FIG. 8), indicating DNA hypomethylation is largely independent of GC therapy in patients with active disease.

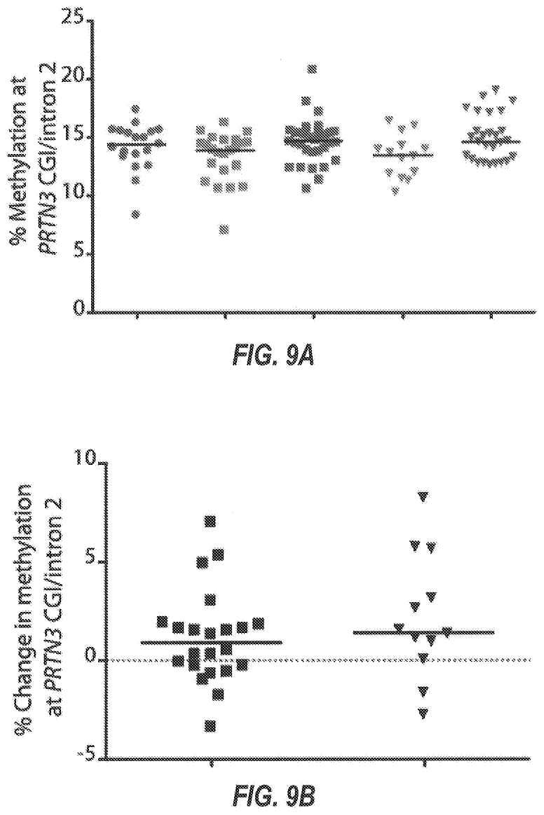

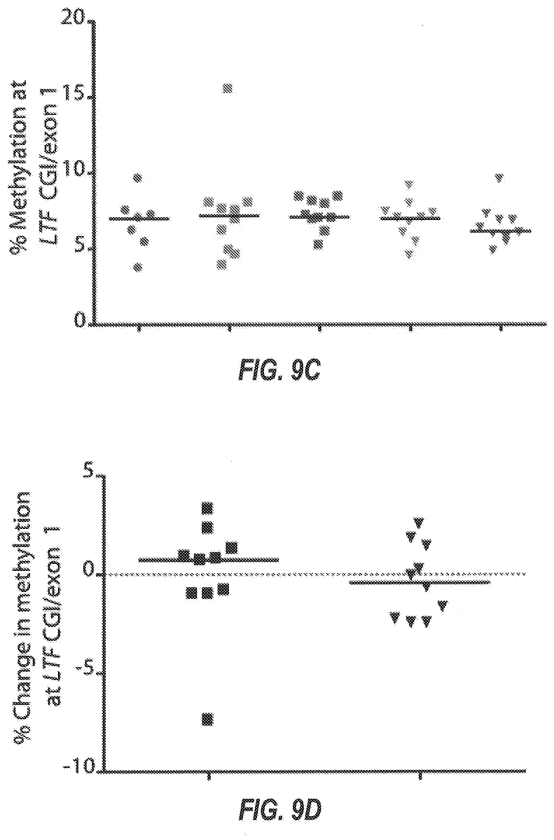

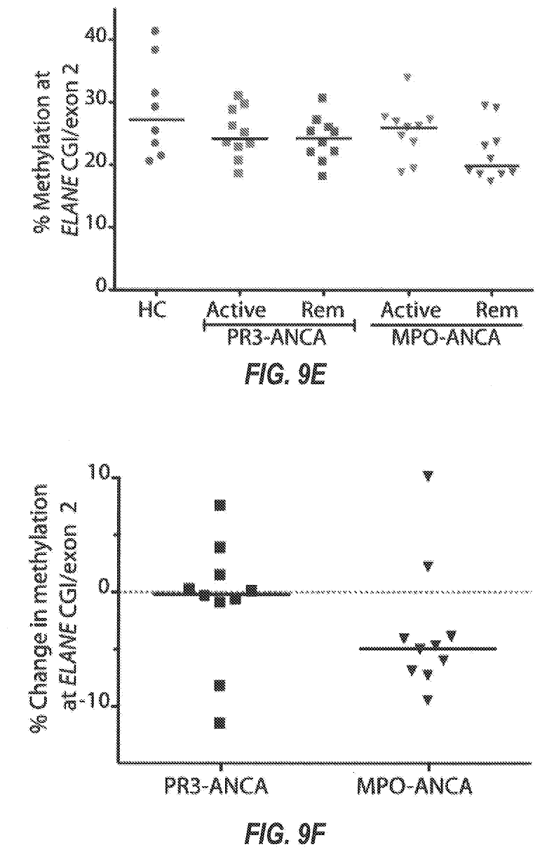

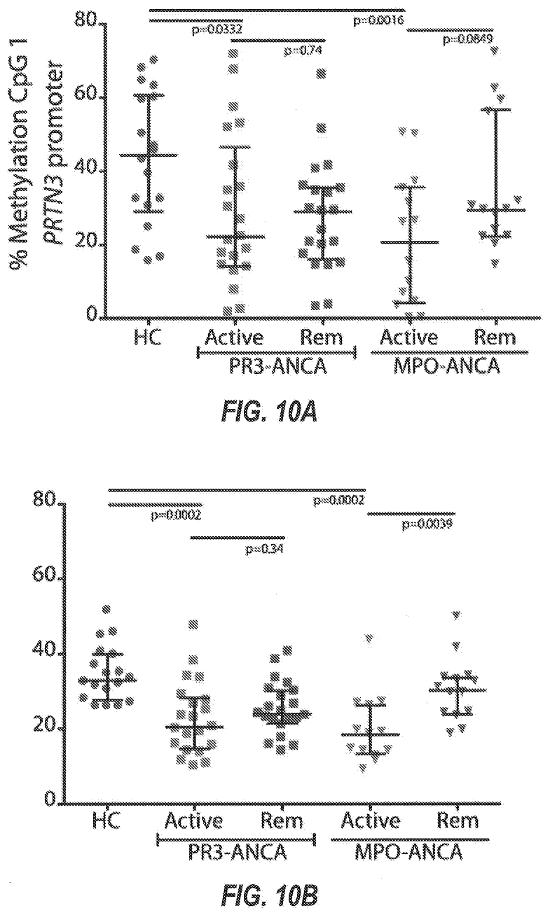

DNA methylation at ELANE, LTF and a CGI overlapping the alternative promoter of PRTN3 was low and not different compared to healthy controls or between patients of different serotypes or disease status (FIGS. 9A-F). We validated a subset of the samples with bisulfite sequencing and found both platforms produced comparable methylation patterns at identical CpGs (FIGS. 10A and 10B). Differences in DNA methylation pattern between the methylation platforms at ELANE are likely due to differences in probe location. DNA methylation at specific loci within MPO and PRTN3 is reduced in patients with active AAV compared to patients in remission and healthy individuals.

Correlation of mRNA Expression with DNA Methylation.

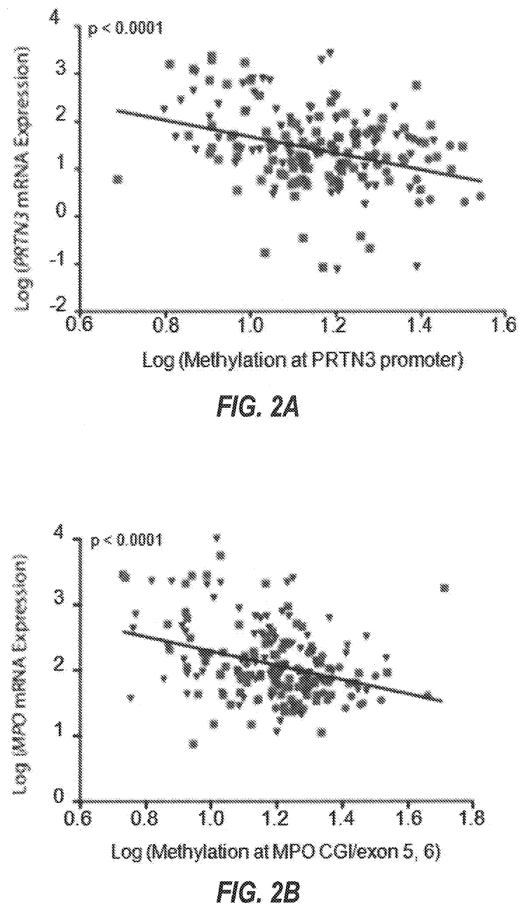

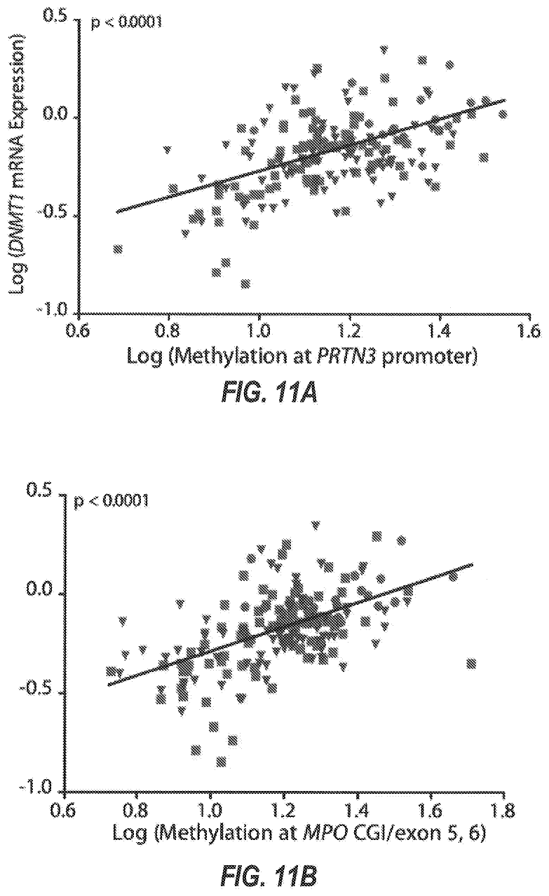

DNMT1 mRNA expression positively correlated with DNA methylation, confirming the association found in the genome-wide DNA methylation data (r=0.4858 at PRTN3 promoter; r=0.5464 at MPO CGI/exon 5-6; p<0.0001) (FIGS. 11A and 11B). MPO and PRTN3 mRNA expression negatively correlated with DNA methylation within MPO and PRTN3 genes (PRTN3 promoter r=−0.2828; MPO CGI/exon 5-6 r=−0.3155; p<0.0001) (FIGS. 2A and 2B). The correlation of cross-sectional measurements suggests that DNA methylation in total leukocytes has a minor influence on expression of these autoantigen genes; however, following patients longitudinally may be more valuable than measuring expression and methylation at a single point in time.

Longitudinal DNA Methylation Studies Uncover Two Distinct Patterns in AAV Patients.

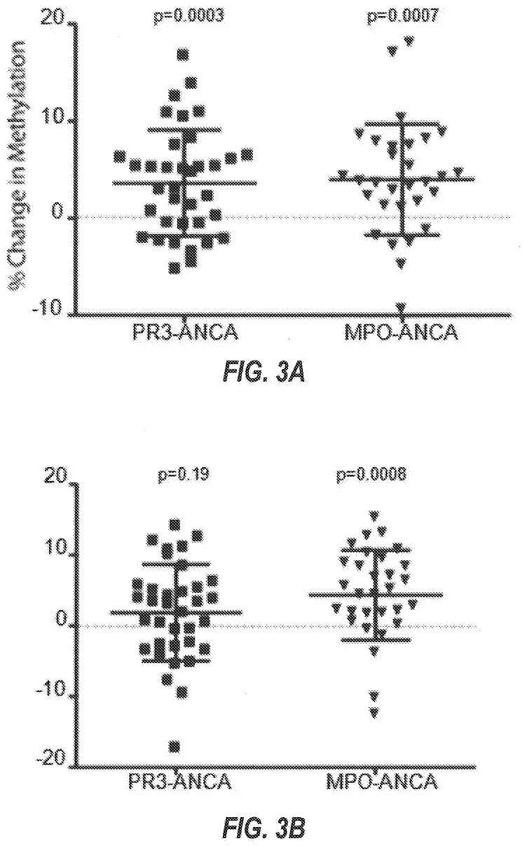

We hypothesized that methylation changes relate to disease status in patients with AAV; to this end, we measured DNA methylation in paired patient samples collected an average of 18 months apart and quantified the difference in methylation (remission minus active methylation) at loci within MPO and PRTN3. At the PRTN3 promoter, the mean change in DNA methylation is comparable between PR3-ANCA patients (3.62%) and MPO-ANCA patients (3.99%) and significantly different from zero (p=0.0003 and 0.0007, respectively) (FIG. 3A). This indicated most patients increased DNA methylation at the PRTN3 promoter upon disease remission, regardless of serotype. At other loci, changes in DNA methylation status depended on serotype. At the MPO CGI/exon 5-6, the mean DNA methylation change in MPO-ANCA patients was similar to that at the PRTN3 promoter (4.36%) but not in PR3-ANCA patients (1.85%) (p=0.0008 and 0.12, respectively) (FIG. 3B). Similarly, changes in DNA methylation at both PRTN3 CGI/exon 5 and MPO CGI/exon 7 were significantly different from zero in MPO-ANCA patients, but not different from PR3-ANCA patients (FIGS. 7C and 7D).

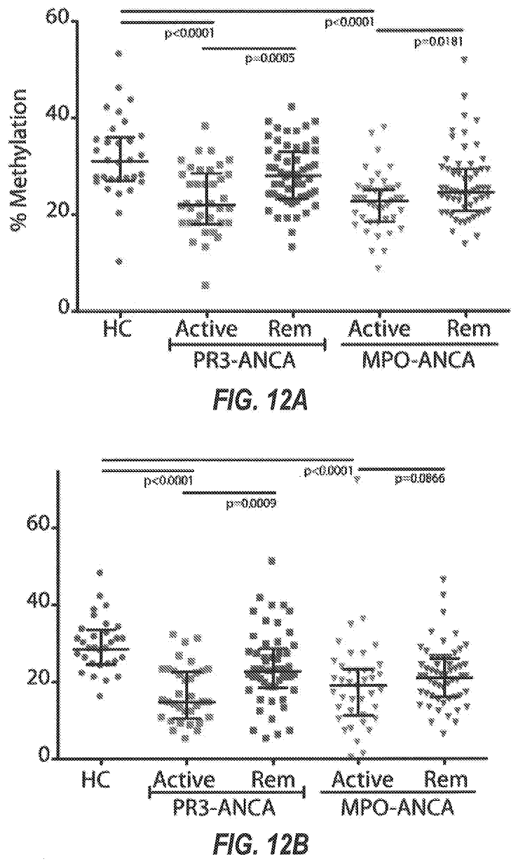

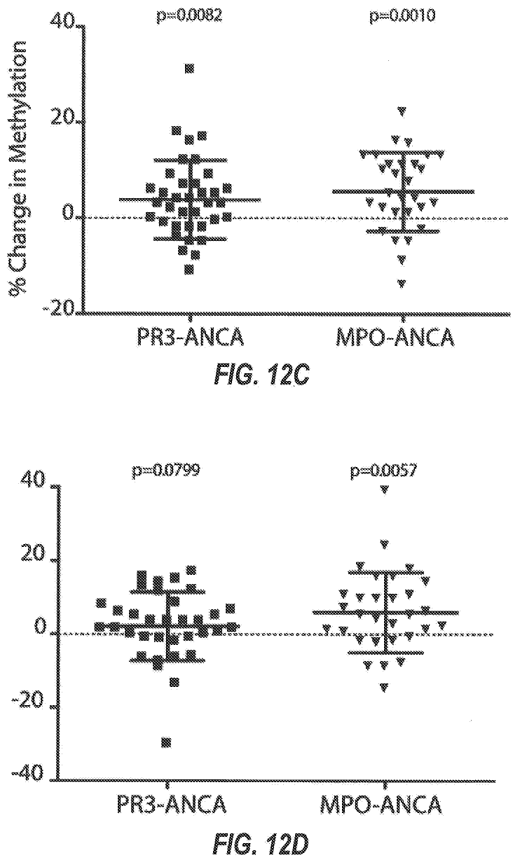

These changes in DNA methylation were based on averaged values of the 13-39 CpGs across an amplicon. Individual CpGs within each locus had either static or dynamic DNA methylation patterns; the patterns at individual dynamic CpGs within the PRTN3 promoter and MPO CGI/exon 5-6 mirror the patterns seen when averaging the CpGs (FIGS. 12A-D). From the longitudinal analysis, we conclude that changes in DNA methylation identified patients who increased DNA methylation when in remission and patients who decreased DNA methylation during remission. Importantly, this segregates patients into two groups where the role of DNA methylation was examined separately.

Autoantigen Gene Expression Correlates with Gene-Specific DNA Methylation in AAV Patients.

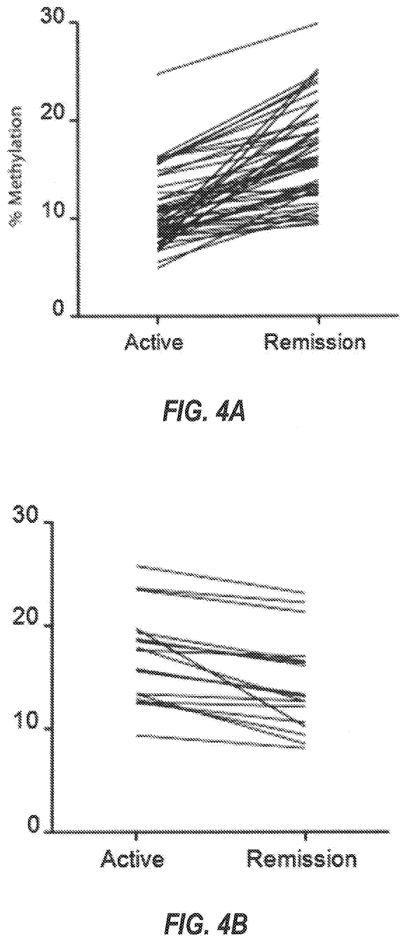

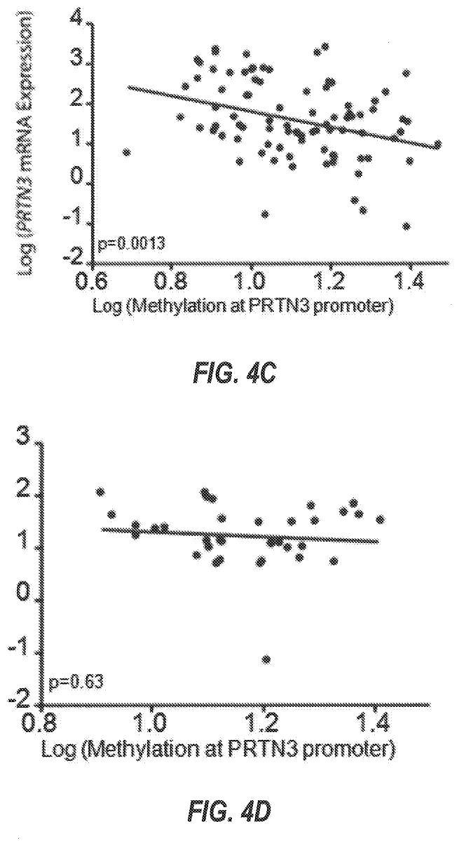

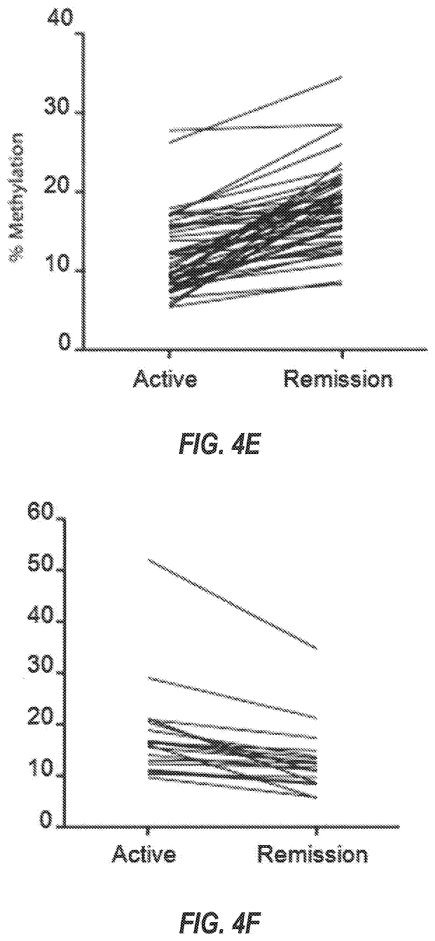

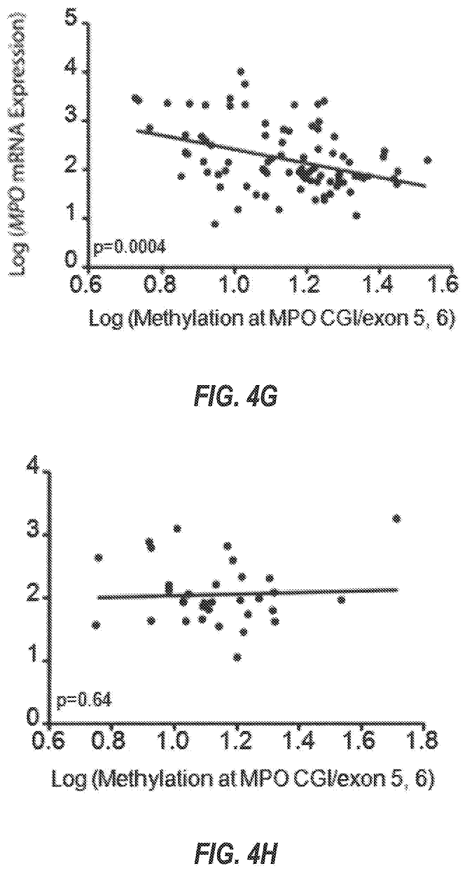

Stratifying paired patients by DNA methylation increase (FIGS. 4A and 4E) or decrease (FIGS. 4B and 4F) revealed a stronger correlation between PRTN3 mRNA expression and DNA methylation in patients with increased DNA methylation at the PRTN3 promoter (r=−0.3390, p=−0.0013) (FIG. 4C) compared to patients with decreased methylation (r=−0.08322, p=0.63) (FIG. 4D). This correlation is also stronger than that seen in the entire cohort (FIG. 2C). The same trend is found at MPO CGI/exon 5-6: DNA methylation correlates with MPO mRNA in patients with increased DNA methylation (r=−0.3735, p=0.0004) while DNA methylation does not correlate with MPO mRNA in patients with decreased methylation (r=−0.08508, p=0.64) (FIGS. 4G and 4H). These data suggest DNA methylation plays a stronger role in regulating expression in patients who exhibit increased gene-specific DNA methylation during disease remission.

Change in DNA Methylation at the PRTN3 Promoter is an Indicator of Relapse in AAV.

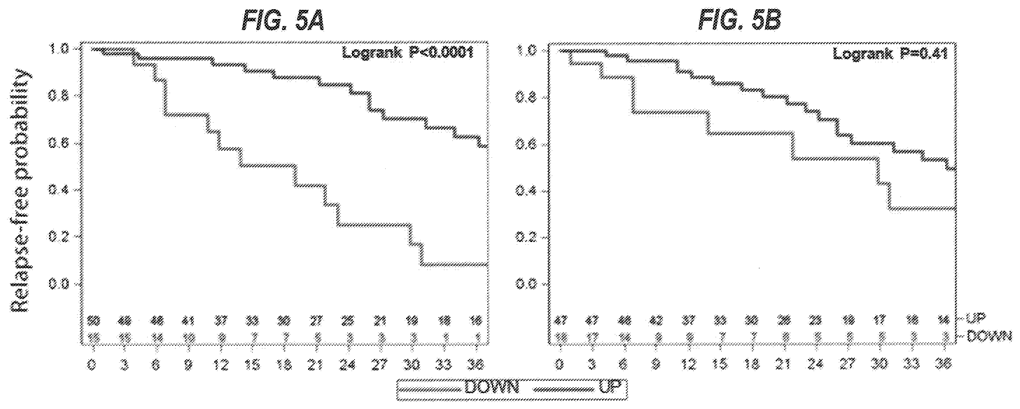

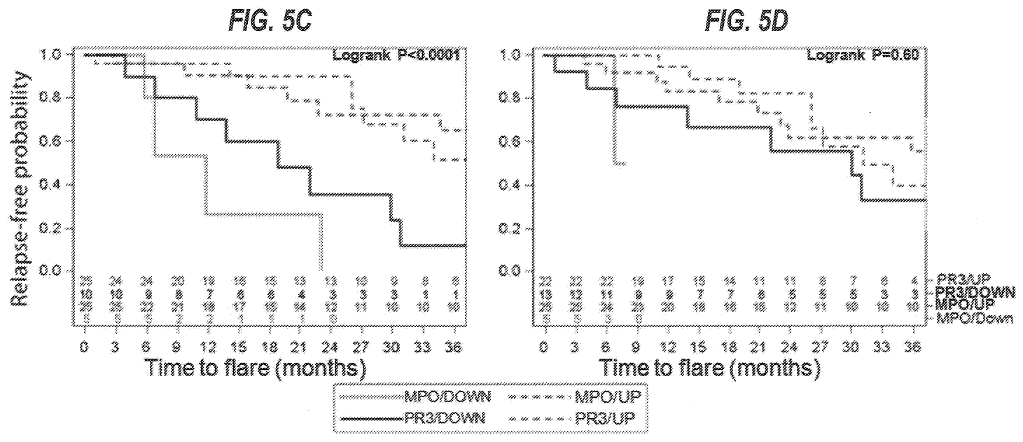

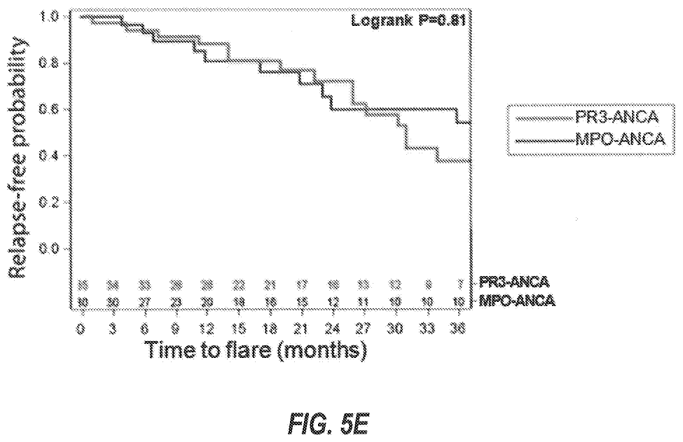

We then questioned if the change in DNA methylation could inform disease prognosis. Within our longitudinal cohort, 65 patients continued to be followed in our clinic. Thirty-four of these patients remain in stable disease remission with a mean clinical follow-up time of 27 months since entering disease remission. Of the paired patients who have relapsed, the average time to relapse was 31 months for those exhibiting increased gene-specific DNA methylation and 16 months for those exhibiting decreased gene-specific DNA methylation. Kaplan-Meier survival analysis demonstrated the change in DNA methylation at the PRTN3 promoter resulted in a higher relapse-free probability among patients (n=50) with increased DNA methylation upon disease remission than among patients (n=15) with decreased DNA methylation (p<0.0001) (FIG. 5A). At MPO CGI/exon 5-6, the relapse-free probability was slightly higher among patients with increased DNA methylation than among patients with decreased methylation, yet not significantly different between the two groups (p=0.41) (FIG. 5B).

To determine if ANCA specificity was responsible for the relapse-free probability, patients with increased or decreased DNA methylation were subdivided by serotype. ANCA serotype did not impact relapse-free probability at the PRTN3 promoter or MPO CGI/exon 5-6, (FIGS. 5C and 5D). Stratifying patients solely on serotype showed similar relapse-free probabilities (p=0.81) (FIG. 5E). This suggests an increase in DNA methylation in disease remission at the PRTN3 promoter is a better indicator of disease prognosis.

Risk of Relapse is Highest in AAV Patients with Decreased DNA Methylation at the PRTN3 Promoter.

Proportional hazard models of time to relapse showed that patients with decreased DNA methylation at the PRTN3 promoter were 4.55 times more likely to relapse (95% CI 2.09, 9.91; p=0.0001). The change in DNA methylation at MPO CGI/exon 5-6 was not predictive of relapse with a hazard ratio of 1.41 (95% CI 0.62, 3.20; p=0.41) (Table 3). When multivariate analyses were performed to control for additional variables, the hazard ratio for relapse among patients that decreased DNA methylation versus patients that increased methylation at the PRTN3 promoter remained significant and did not change from the univariate analysis (Table 3). Based on this analysis, the change in DNA methylation at the PRTN3 promoter indicates the likelihood of relapse among patients with AAV.

In this cohort, polymorphonuclear cell (PMN) counts were available for 22 patient pairs. In this small subset of patients, we found the same association between PMN count and DNA methylation at both the PRTN3 promoter and MPO CGI/exon 5-6; yet only the change in DNA methylation at the PRTN3 promoter was found to be predictive of stable remission. These preliminarily findings suggest PMN count does not explain our observation that a change in DNA methylation at the PRTN3 promoter predicts relapse probability.

Since individual CpGs show dynamic changes, we tested whether changes in DNA methylation at dynamic CpGs in MPO CGI/exon 5-6 and the PRTN3 promoter associated with different relapse probabilities or likelihood of relapse. Of the individual CpGs interrogated, the change in DNA methylation at CpG 13 in the PRTN3 promoter was predictive of relapse with a hazard ratio of 3.43 (95% CI 1.56, 7.56; p=0.0022). This pinpoints a single cytosine residue where a change in DNA methylation may be prognostic.

In the present invention, we tested whether differential gene-specific DNA methylation occurs during the natural history of AAV and is associated with probability of relapse. We measured DNA methylation in paired patients with AAV during active disease and remission because preliminary data indicated DNMT1 mRNA expression was reduced in AAV patients, and to determine whether differential DNA methylation is associated with disease state. We confirmed DNMT1 mRNA was reduced in active patients and increased when patients were in remission. In a genome-wide analysis of differential DNA methylation between patients with active disease and healthy individuals, we found that only 881 and 953 genes had greater decreases in methylation than what was detected at MPO and PRTN3, respectively. Although other genes had greater differences in the DNA methylation, the differential DNA methylation at MPO and PRTN3 appears to be specific because the degree of differential DNA methylation at ELANE, another neutrophil granule gene with a similar expression pattern to MPO and PRTN3, resulted in 1591 genes with a greater decrease in DNA methylation.

We found DNA methylation was dynamic. The change in DNA methylation from active disease to remission revealed two groups of patients: those that increase methylation, and those that decrease methylation. This dichotomy has striking consequences. First, more PR3-ANCA patients decrease DNA methylation at PRTN3 CGI/exon 5 and MPO CGI/exon 5-6 than MPO-ANCA patients. This suggests that there are epigenetic differences in addition to genetic differences between MPO-ANCA and PR3-ANCA patients. Second, autoantigen gene expression correlated with DNA methylation among patients with increased methylation, implying DNA methylation may regulate transcription of autoantigen genes among these patients. Finally, most intriguing is the finding that DNA methylation changes indicated the likelihood a patient would relapse. At the PRTN3 promoter, a decrease in DNA methylation upon disease remission indicates a higher probability a patient will relapse in the future, regardless of ANCA serotype, compared to patients that increased methylation in disease remission. The DNA methylation changes at the PRTN3 promoter in MPO-ANCA patients are consistent with our previous data showing MPO and PRTN3 genes are coordinately expressed and share changes in histone methylation, suggesting the genes are regulated similarly in AAV patients with either MPO-ANCA or PR3-ANCA.

While DNA methylation at PRTN3 promoter and MPO CGIs correlated with MPO and PRTN3 expression among patients with increased DNA methylation, the only prognostic value for DNA methylation was found in the promoter of PRTN3. Within the PRTN3 promoter we were able to identify an individual CpG that was as predictive as the mean DNA methylation change. This CpG (13) resides within a CG element previously characterized as critical for PRTN3 expression and recognized by a protein of approximately 40 kDa. DNA methylation at this CpG could disrupt transcription factor binding; previous studies in autoimmunity have demonstrated the importance of methylation at individual CpGs. The lack of a predictive value of DNA methylation at the CGIs could indicate transcription is regulated by another mechanism at these sites. Polycomb-like 3/PHF19 promotes binding to CGIs of PRC2, which is responsible for H3K27 methylation. This epigenetic silencing mark was reduced at MPO and PRTN3 genes in patients with active AAV. Therefore, a parsimonious explanation is that either DNA methylation or H3K27me3 can act at CGIs within MPO and PRTN3 to regulate their expression; while at the PRTN3 promoter, DNA methylation is the dominant transcriptional control mechanism.

This study shows that a change in DNA methylation at the PRTN3 promoter is sufficient to indicate the likelihood of relapse.

Although up to 90% of AAV patients achieve remission through induction therapy, many patients experience a relapse, making insights into disease prognosis vital to patient care. We studied a longitudinal cohort of heterogeneous AAV patients, with a MPA or GPA diagnosis and MPO- or PR3-ANCA serotypes, alongside their corresponding clinical information to test whether a change in DNA methylation may inform disease state and prognosis. Currently clinical criteria distinguish the disease status of patients; however, we show DNA methylation changes at MPO and PRTN3 further stratify disease remission status. In patients with ANCA-associated vasculitis, DNA methylation changes at the PRTN3 promoter are a potential indicator of stable remission.

Study Design.

The objective of this study was to characterize the DNA methylation patterns in paired patients with AAV through states of disease activity and remission. This was an observational study of DNA methylation changes, both globally and at specific loci. AAV patients were enrolled at UNC-Chapel Hill clinics and followed in the Glomerular Disease Collaborative Network (GDCN). Patients and healthy volunteers were recruited, according to the guidelines of the Institutional Review Board (IRB study #97-0523) by the University of North Carolina Office of Human Research Ethics. Study subjects gave informed, written consent and participated according to UNC IRB guidelines. We stopped collecting patient samples once we achieved a statistically significant difference between active patients and healthy controls and a statistically significant hazard ratio for patients with decreased DNA methylation at the PRTN3 promoter. We used a power analysis to calculate the sample size (100) necessary to achieve a reliable measurement of DNA methylation changes in patients with AAV. Preliminary data from a smaller sample size was used to recalculate our power analysis and change our sample size to 80 unique patients. De-identified patient and healthy control samples were assigned randomly to plates for DNA methylation analysis and run in duplicate on separate plates. Paired samples from the same patient were rarely run on the same plate or in the same batch. Generally, samples were processed in the order that they were retrieved from the freezer or the order in which the patients presented at clinic. Investigators who quantified the results were blinded with regard to the type of patient or control being analyzed.

Patient Cohort.

Patients were diagnosed according to the Chapel Hill Consensus Conference. ANCA serotypes were determined by indirect immunofluorescence and antigen-specific PR3 and MPO enzyme-linked immune-absorbent assays (ELISA). Disease activity was determined by the 2003 Birmingham Vasculitis Activity Score (BVAS) in conjunction with clinical signs of activity. In this study, patients with a BVAS of 0 and no clinical or laboratory evidence of active disease were considered to be in remission. Active disease was defined as a BVAS >0 with clinical and/or laboratory evidence of disease. A total of 82 patients with AAV (Table 4) and 32 healthy controls (Table 5) were chosen for this study based on the availability of paired active/remitting disease samples, clinical data and laboratory data. Patients with suspected or confirmed drug-induced forms of AAV that were ANCA negative by ELISA, or had overlapping disease were excluded. Patients taking known epigenetic modifiers were also excluded from this study. Patient demographics were similar between healthy controls and AAV patients with regard to age, gender and race.

AAV patients were selected for this study based on the availability of total leukocyte DNA and RNA collected at a point of clear disease activity or disease remission (on or off therapy). In addition to the presence of DNA and RNA samples for each patient, we also ensured there was adequate clinical information including BVAS and a list of immunosuppressant therapies the patient was taking at the time of each sample collection. For longitudinal studies we selected patients for whom samples were available at a time of disease activity and remission and were, on average, 18 months apart. The range of time between the active sample and the remitting sample for the 65 pairs in the longitudinal study is 2-113 months (MPO-ANCA pairs 2-52 months; PR3-ANCA pairs 2-113 months). The mean time between samples was indeed 18 months (MPO-ANCA 16 months; PR3-ANCA 19 months) and the median time between samples was 13 months (MPO-ANCA 12 months; PR3-ANCA 16 months); the standard deviation is 19.9 (MPO-ANCA 14.3; PR3-ANCA 24.0). Patients in this study did not have multiple flares between measurements and had been in clinical disease remission an average of eight months before the second measurement was taken. These criteria were established prospectively. No outliers have been excluded from this study.

RNA and DNA Isolation from Total Leukocytes.

Total circulating leukocyte RNA was obtained from EDTA-treated whole blood using RNA STAT-60 (Tel-Test “B”, Friendswood, Tex., USA). Qiagen reagents (Chatsworth, Calif.) including the RNeasy Mini Kit and RNase-Free DNase Set, were used to isolate RNA from total leukocytes. Sodium citrate-treated whole blood was used to isolate DNA from total leukocytes. For DNA isolation we use Cell Lysis Solution, Protein Precipitation Solution, DNA Hydration Solution (all Puregene Accessories, available through Qiagen) and RNase A from bovine pancreas (Sigma-Aldrich, St. Louis, Mo.).

DNA Methylation Studies.

Total leukocyte DNA was bisulfite-converted in duplicate using the EZ DNA methylation kit (Zymo Research, Orange, Calif.). Bisulfite-treated DNA samples were used in three separate platforms for measuring DNA methylation: EpiTyper MassARRAY (Agena, La Jolla, Calif.), bisulfite sequencing (Zymo Research) and Illumina Infinium HumanMethylation450 BeadChip (Illumina, Inc.).

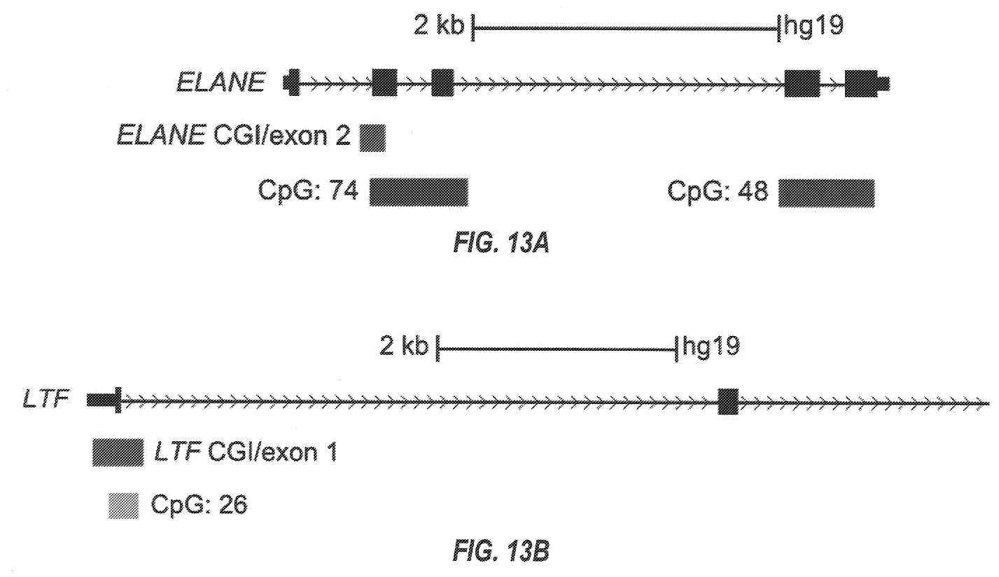

Targeted MALDI-TOF mass spectrometry (EpiTYPER®, Agena Bioscience) was carried out at seven amplicons within MPO, PRTN3, LTF and ELANE (FIGS. 1B and 1C; FIGS. 13A and 13B). These amplicons cover CpGs that were also interrogated using the genome-wide DNA methylation platform. Primer pairs were designed using EpiDesigner software (www.epidesigner.com) (Table 6). A cohort of 82 AAV patients and 32 healthy individuals were run on this platform, in duplicate. In accordance with the standard protocol and following amplification of 650 ng of bisulfite-converted DNA, in duplicate, the PCR products underwent the SAP treatment and T-cleavage reaction in preparation for quantitative analysis of DNA methylation. Mean DNA methylation for each sample was measured by averaging the CpGs in each individual amplicon and the average DNA methylation value of the replicates was reported.

Targeted bisulfite sequencing for DNA methylation analysis was done on a replication cohort of 77 patient samples and 19 samples from healthy individuals (96 samples) at six of the same loci studied interrogated using the Agena platform. Sixteen primers were designed, synthesized (Integrated DNA Technologies) and validated by Zymo Research (Table 7). Targeted amplification of these samples was performed according to manufacturer's protocol (Zymo Research and Fluidigm).

Illumina Infinium HumanMethylation450 BeadChips were used to analyze DNA methylation on a genome-wide scale in ten longitudinally paired AAV patients and four age-matched healthy individuals (24 samples). This platform allows for interrogation of >485,000 methylation sites per sample, covering 99% of RefSeq genes. After bisulfite treatment, the Mammalian Genotyping Core at UNC-Chapel Hill performed the remaining assay steps following the specifications and using the reagents supplied by the manufacturer.

Taqman mRNA Expression Studies.

Quantitative detection of DNMT1 mRNA levels from patient samples was determined as relative to three healthy control samples run on each plate. Quantitative detection of MPO and PRTN3 mRNA levels from patient samples was determined using a standard curve. The standard curve for MPO mRNA levels was generated using HL60 cells, a cell positive for MPO mRNA, diluted with Jurkat cells, a cell line negative for MPO mRNA. The standard curve for PRTN3 mRNA levels was generated using THP-1 cells, a cell positive for PTRN3 mRNA, diluted with Jurkat cells, a cell line negative for PRTN3 mRNA. MPO and PRTN3 mRNA levels for patients and healthy donor samples were determined by 2^{-ΔΔCt} calculations and expressed relative to standard curves. Primers and probes for MPO and PRTN3 can be found in Table 6. Cytochrome c oxidase (COX5B) was used as mRNA internal control. Primers and probes were purchased from Applied Biosystems (Applied Biosystems, Foster City, Calif.) and Integrated DNA Technologies, Inc. (Coralville, Iowa). Quantitative RT-PCR assays were performed on an ABI PRISM 7900HT sequence detection system using the TaqMan EZ RT-PCR kit (Applied Biosystems) (Yang, et al., manuscript submitted).

Genome-Wide DNA Methylation Analysis.

Analysis of the Illumina Infinium HumanMetylation450 BeadChip array was performed in R using the minfi package and the UCSC hg19 knownGene genome annotation. The red and green intensities were converted to methylation data using background correction and SWAN normalization. Probes with SNPs at the CpG interrogation site or at the single base extension were omitted from analysis. Beta values were logit transformed to draw boxplots and compare DNA methylation at specific sites. These boxplots where made using the default R settings for the boxplot function (FIG. 6).

Statistical Analysis.

Comparisons between two independent groups were done using Wilcoxon rank sum test. Bonferroni corrections were used in situations with multiple comparisons between groups. Mean DNA methylation was measured by averaging the CpGs in each individual amplicon. Methylation at individual CpGs was found to be either static or dynamic in a pattern mirroring that shown in the mean DNA methylation. Log transformed correlation for DNA methylation and the expression of autoantigen genes was done by Spearman correlation coefficients. Kaplan-Meier curves with log rank tests were used to display and compare relapse-free survival times. These curves were used to evaluate the proportional hazards assumption, and then proportional hazards models were used to model the effect of DNA methylation (PRTN3 Promoter, PRTN3 Promoter CpG13, or MPO CGI/exon 5-6) on time to relapse. Potential confounders were modeled controlling for DNA methylation with one additional variable at a time. Univariate predictors of flare are reported as hazards ratios and 95% confidence intervals with a two-sided P-value of 0.05 or less considered statistically significant. DNA methylation change between the active and remission groups was analyzed with signed-rank test on all available longitudinal samples within patients (n=65) and then limited to the first pair collected chronologically for individuals (n=60). Results were almost exactly the same, thus results displayed include the full number of pairs. All analyses were done by R and SAS 9.4 (SAS Institute, Cary, N.C., USA).

The foregoing is illustrative of the present invention, and is not to be construed as limiting thereof. The invention is defined by the following claims, with equivalents of the claims to be included therein.

TABLE 1
Demographics and clinical parameters of patients and healthy individuals included in this study

| | | Healthy individuals | PR3-ANCA | MPO-ANCA patients |
|---|---|---|---|---|
| Variables | | (32) | patients (42) | (40) |
| Age | N | 32 | | |
| | Mean ± SD | 52.78 ± 14.61 | | |
| | Median (IQR) | 56.50 (51.00, 59.00) | | |
| Sex | Female | 16 (50.00%) | 17 (40.48%) | 19 (47.50%) |
| | Male | 16 (50.00%) | 25 (59.52%) | 21 (52.50%) |
| Race | Caucasian | 27 (84.38%) | 35 (83.33%) | 32 (80.00%) |
| | Other | 5 (15.63%) | 7 (16.67%) | 8 (20.00%) |
| Diagnosis | EGPA | | 0 (0.00%) | 2 (5.00%) |
| | GPA | | 27 (64.29%) | 8 (20.00%) |
| | LIM | | 0 (0.00%) | 12 (30.00%) |
| | MPA | | 15 (35.71%) | 18 (45.00%) |
| Sample number | | | 98 | 86 |
| | Active Samples | | 42 | 35 |
| | Active samples taking GC | | 18 | 16 |
| | Remitting samples | | 56 | 51 |
| | Rem. samples taking GC | | 14 | 9 |
| Active, new onset | | | 10 (10.20%) | 11 (12.79%) |
| Long-term remission, off | | | 12 (12.24%) | 18 (20.93%) |
| therapy | | | | |
| Active sample age (years) | N | | 42 | 35 |
| | Mean Age (years) ± SD | | 56.21 ± 14.53 | 53.51 ± 20.22 |
| | Median (IQR) | | 57.00 (50.00, 67.00) | 57.00 (33.00, 69.00) |
| Remission sample age | N | | 56 | 51 |
| (years) | Mean ± SD | | 57.57 ± 16.75 | 52.47 ± 18.99 |
| | Median (IQR) | | 59 (46.50, 71.00) | 54.00 (38.00, 66.00) |
| Active sample BVAS | N | | 42 | 35 |
| | Mean ± SD | | 8.07 ± 6.91 | 9.49 ± 6.42 |
| | Median (IQR) | | 6.00 (2.00, 13.00) | 8.00 (6.00, 12.00) |
| Remission sample BVAS | N | | 56 | 51 |
| | Mean ± SD | | 0 ± 0 | 0.11 ± 0.43 |
| | Median (IQR) | | 0.00 (0.00, 0.00) | 0.00 (0.00, 0.00) |

PR3, proteinase 3;
MPO, myeloperoxidase;
MPA, microscopic polyangiitis;
GPA, granulomatosis with polyangiitis;
EGPA, eosinophilic granulomatosis with polyangiitis;
Lim, renal limited vasculitis;
BVAS, Birmingham Vasculitis Activity Score;
GC, glucocorticoids.

TABLE 2
Number of genes with larger differential
methylation than at MPO and PRTN3

| | Largest mean M- | Genes with | | |
|---|---|---|---|---|
| | value difference | larger mean M | | |
| | (Active v HC) | value difference | Unique genes | p-value |
| MPO | −1.34 | 881 | 19654 | 0.045 |
| PRTN3 | −1.31 | 953 | 19654 | 0.048 |

TABLE 3
Hazard ratios for the association of relapse with change in DNA methylation at PRTN3 and MPO, and HR
controlled for individual variables.

| | | PRTN3 Promoter | | PRTN3 Promoter CpG13 | | MPO CGI/exon 5, 6 | |
|---|---|---|---|---|---|---|---|
| | Controlled variables | HR (95% CI) | p-value* | HR (95% CI) | p-value* | Hazards ratio | p-value* |
| Methylation | | 4.55 (2.09, 9.91) | 0.0001 | 3.43 (1.56, 7.56) | 0.0022 | 1.41 (0.62, 3.20) | 0.41 |
| | Serotype¹ | 1.43 (0.64, 3.21) | 0.38 | 1.29 (0.59, 2.86) | 0.52 | 1.07 (0.46, 2.49) | 0.88 |
| | Age² | 0.99 (0.97, 1.01) | 0.36 | 0.99 (0.97, 1.01) | 0.28 | 0.99 (0.97, 1.02) | 0.59 |
| | Diagnosis³ | 2.16 (0.99, 4.75) | 0.0541 | 1.97 (0.90, 4.31) | 0.0906 | 2.39 (1.09, 5.24) | 0.0289 |
| | PRTN3 expression change⁴ | 1.60 (0.62, 4.15) | 0.34 | 1.39 (0.54, 3.57) | 0.50 | 1.08 (0.43, 2.73) | 0.87 |
| | MPO expression change⁴ | 0.99 (0.39, 2.54) | 0.99 | 0.85 (0.34, 2.14) | 0.73 | 0.68 (0.27, 1.70) | 0.41 |
| | Glucocorticoid Therapy⁵ | | | | | | |
| | Active samples | 0.93 (0.43, 1.99) | 0.84 | 0.91 (0.42, 1.95) | 0.80 | 0.74 (0.35, 1.55) | 0.42 |
| | Remission samples | 1.44 (0.50, 4.12) | 0.50 | 0.98 (0.37, 2.60) | 0.97 | 0.82 (0.31, 2.16) | 0.68 |
| | Organ involvement⁶ | | | | | | |
| | Lung | 0.97 (0.46, 2.07) | 0.94 | 1.10 (0.52, 2.32) | 0.81 | 1.07 (0.51, 2.28) | 0.85 |
| | Upper Respiratory | 1.52 (0.70, 3.27) | 0.29 | 1.67 (0.78, 3.56) | 0.18 | 1.75 (0.81, 3.77) | 0.15 |
| | Kidney | 0.93 (0.31, 2.86) | 0.91 | 0.73 (0.26, 2.00) | 0.54 | 1.53 (0.53, 4.41) | 0.43 |

*P values were calculated using each hazard model of relapse controlled for the variables listed.
¹MPO-ANCA (n = 30) compared to PR3-ANCA patients (n = 35)
²Age at time of first sample (usually active)
³GPA (n = 27) compared to all grouped MPA (n = 29), Renal Limited (n = 7) and EGPA (n = 2)
⁴Expression at time of disease remission, subtracted from expression at time of disease activity; positive changes compared to negative changes
⁵Samples on glucocorticoid therapy (GC) (active n = 34; remission n = 23) compared to those not taking GC (active n = 43; remission n = 84)
⁶Lung involvement (n = 33); upper respiratory involvement (n = 36); kidney involvement (n = 50)

TABLE 4
Healthy individual demographics: Age, gender, race and mRNA
expression of PRTN3, MPO and DNMT1 for each
of 32 healthy controls

| | | | | mRNA Expression | | |
|---|---|---|---|---|---|---|
| Sample | Age | Sex | Race | PRTN3 | MPO | DNMT1 |
| 1 | 47 | M | W | NA | NA | NA |
| 2 | 57 | M | W | 16.05 | 105.72 | 1.22 |
| 3 | 52 | M | W | NA | NA | NA |
| 4 | 66 | F | A | NA | NA | NA |
| 5 | 52 | F | W | NA | NA | NA |
| 6 | 22 | F | W | 2.09 | 33.12 | 1.84 |
| 7 | 56 | F | W | 28.29 | 66.92 | 0.75 |
| 8 | 51 | F | W | NA | NA | NA |
| 9 | 59 | F | W | NA | NA | NA |
| 10 | 69 | M | W | 19.56 | 66.98 | 0.84 |
| 11 | 67 | M | W | 1.85 | 38.93 | 1.21 |
| 12 | 65 | M | W | 51.72 | 102.3 | 0.81 |
| 13 | 51 | F | W | 28.64 | 29.89 | 1.18 |
| 14 | 59 | F | B | 9.69 | 33.14 | 0.71 |
| 15 | 58 | F | W | NA | NA | NA |
| 16 | 57 | F | W | 5.56 | 38.14 | 0.97 |
| 17 | 57 | F | W | 14.79 | 77.65 | 0.9 |
| 18 | 54 | M | W | 45.4 | 118.73 | 1.49 |
| 19 | 22 | M | A | NA | NA | NA |
| 20 | 22 | M | W | 8.07 | 25.14 | 0.79 |
| 21 | 58 | F | W | 11.29 | 42.73 | 0.87 |
| 22 | 26 | M | B | 2.46 | 25.03 | 1.03 |
| 23 | 22 | M | A | NA | NA | NA |
| 24 | 58 | F | W | 26.67 | 24.77 | 0.7 |
| 25 | 56 | F | W | 1.81 | 28.46 | 0.86 |
| 26 | 77 | M | W | 15.06 | 34.61 | 0.84 |
| 27 | 69 | M | W | 13.74 | 58.12 | 0.56 |
| 28 | 56 | M | W | 10.71 | 35.89 | NA |
| 29 | 64 | F | W | NA | NA | NA |
| 30 | 56 | M | W | 33.21 | 175.38 | 0.76 |
| 31 | 47 | M | W | 50.53 | 156.82 | 0.92 |
| 32 | 57 | F | W | NA | NA | 0.85 |

TABLE 5
Complete demographic and clinical data for patient cohort: Eighty-two patients and 184 unique samples collected alongside
clinical data and mRNA expression for PRTN3, MPO and DNMT1

| | | | | | | | Disease | | | | | | | | | |
|---|---|---|---|---|---|---|---|---|---|---|---|---|---|---|---|---|
| | | | | | | | Status | | ANCA | | | | | WBC | PMN | |
| | Sam- | Diag- | | | | Sero- | *=new | | Titer | | mRNA Expression | | | Ct | Ct | |
| Patient | ple | nosis | Age | Sex | Race | type | onset | BVAS | PR3 | MPO | PRTN3 | MPO | DNMTI | (×10⁹ cells/L) | | Therapy |
| 1 | 1a | MPA | 23 | F | W | MPO | Active | 18 | 2.2 | 47.6 | 4.90 | 70.31 | NA | 5.9 | 4 | MMF |
| | 1b | | 26 | | | | Remission | 0 | 2 | 21.4 | 1.65 | 26.16 | 0.58 | 5.8 | NA | MMF |
| | 1c | | 29 | | | | Active | 6 | 2.1 | 29.5 | 3.18 | 10.78 | 0.52 | 5.9 | NA | None |
| | 1d | | 29 | | | | Remission | 2 | NA | NA | 4.00 | 35.03 | 0.39 | 5.6 | 3.8 | MMF |
| 2 | 2a | Lim | 53 | F | W | MPO | *Active | 10 | 3.4 | 147.1 | 28.47 | 115.91 | 1.32 | 6.5 | 4.5 | CYIV |
| | 2b | | 55 | | | | Remission | 0 | 4.5 | 72.6 | 12.75 | 65.72 | 0.66 | NA | NA | AZA |
| 3 | 3a | Lim | 57 | F | B | MPO | Active | 12 | 2.7 | 85.8 | NA | NA | NA | 11.3 | NA | MPD; |
| | | | | | | | | | | | | | | | | RITUX |
| | 3b | | 58 | | | | Remission | 0 | 3.2 | 3.5 | 3.97 | 42.60 | 2.17 | 2.7 | 1.7 | CS |
| | 3c | | 60 | | | | Remission | 0 | 3.6 | 139.2 | 3.62 | 19.85 | 0.46 | NA | NA | None |
| 4 | 4a | Lim | 78 | M | W | MPO | *Active | 12 | 3.6 | 35.9 | 23.56 | 36.52 | 0.29 | 8.9 | 7.4 | MPD |
| | 4b | | 78 | | | | Remission | 0 | NA | NA | 30.23 | 110.52 | 0.66 | NA | NA | MMF |
| | 4c | | 80 | | | | Remission | 0 | NA | NA | 186.72 | 741.66 | 0.86 | NA | NA | None |
| 5 | 5a | EGPA | 62 | M | W | MPO | *Active | 24 | 5.9 | 21.5 | 211.86 | 581.61 | 0.43 | 13.2 | 11.7 | CS |
| | 5b | | 66 | | | | Remission | 0 | 3.1 | 5.9 | NA | NA | NA | 6.6 | 5.2 | MMF; CS |
| 6 | 6a | MPA | 68 | M | W | MPO | Remission | 0 | 3.1 | 27.2 | 23.68 | 247.81 | 0.59 | NA | NA | MMF |
| | 6b | | 69 | | | | Active | 3 | 2.1 | 53 | 110.45 | 633.26 | 1.1 | 6 | NA | RIUX; |
| | | | | | | | | | | | | | | | | MMF |
| 7 | 7a | Lim | 87 | M | W | MPO | Remission | 0 | 1.9 | 70.4 | 35.66 | 45.86 | 1.35 | 6.1 | 2.4 | RITUX |
| | 7b | | 88 | | | | Active | 6 | 2.1 | 97.1 | 41.63 | 223.74 | 0.81 | 5.9 | 2.7 | None |
| | 7c | | 88 | | | | Remission | 0 | NA | NA | 46.72 | 144.44 | 0.9 | 5.4 | NA | RITUX |
| 8 | 8a | MPA | 58 | M | B | MPO | *Active | 22 | 3.6 | 36.6 | 12.22 | 83.60 | 0.35 | 11.3 | 10.1 | MPD |
| | 8b | | 58 | | | | Remission | 0 | NA | NA | 378.77 | 2400.50 | 0.93 | 12.2 | 9.3 | CYIV |
| 9 | 9a | MPA | 17 | M | W | MPO | Active | 6 | NA | NA | 336.62 | 2070.72 | 1.19 | 6.4 | 4.6 | CS; CYIV; |
| | | | | | | | | | | | | | | | | RITUX |
| | 9b | | 17 | | | | Remission | 0 | NA | NA | 11.97 | 143.00 | 1.39 | 4.9 | 2.7 | RITUX; |
| | | | | | | | | | | | | | | | | AZA |
| | 9c | | 19 | | | | Remission | 0 | NA | NA | 18.46 | 171.66 | 1.29 | 4.9 | NA | RITUX; |
| | | | | | | | | | | | | | | | | AZA |
| 10 | 10a | MPA | 46 | M | W | MPO | Active | 5 | 3 | 32.6 | 1005.43 | 670.70 | 0.48 | 7.3 | 6.7 | CS; CYPO |
| | 10b | | 47 | | | | Remission | 0 | 2.2 | 4.6 | 82.56 | 202.39 | 0.78 | NA | NA | None |
| 11 | 11a | Lim | 72 | M | W | MPO | Active | 6 | 1.9 | 72 | 577.54 | 1913.73 | 0.73 | 9.3 | 8.2 | CS; CYIV |
| | 11b | | 75 | | | | Remission | 0 | 2.9 | 35.1 | 19.08 | 101.04 | 0.61 | 5.1 | 3.4 | None |
| 12 | 12a | MPA | 59 | F | W | MPO | *Active | 8 | 1.6 | 34.4 | 151.86 | 631.38 | NA | NA | NA | CS; CYPO |
| | 12b | | 60 | | | | unclear | 2 | 1.5 | 16.5 | 2.69 | 58.03 | 0.55 | 5.3 | NA | MMF |
| | 12c | | 63 | | | | Remission | 0 | 2.5 | 21.2 | 3.52 | 82.41 | 0.36 | NA | NA | None |
| 13 | 13a | Lim | 51 | M | W | MPO | Active | 6 | 3.4 | 60.6 | 18.47 | 89.13 | 0.5 | 9.8 | 7.5 | MMF; CyA |
| | 13b | | 56 | | | | Remission | 0 | 13.3 | 76.2 | 42.07 | 50.62 | 0.52 | NA | NA | None |
| 14 | 14a | MPA | 37 | F | W | MPO | Active | 0 | 2.7 | 20.1 | 742.25 | 2129.74 | 0.47 | 10.2 | 9.1 | CS; CYIV |
| | 14b | | 40 | | | | Remission | 0 | 2.7 | 21.7 | 44.60 | 174.36 | 0.47 | 5.6 | NA | MMF |
| 15 | 15a | GPA | 74 | M | W | MPO | Active | 3 | 1.6 | 24.6 | 43.55 | 68.94 | NA | 3.8 | NA | MPD |
| | 15b | | 75 | | | | Remission | 0 | NA | NA | 22.55 | 96.26 | 0.61 | 6.2 | 3.9 | MMF |
| 16 | 16a | MPA | 26 | F | W | MPO | *Active | 16 | 2.6 | 42.7 | 61.45 | 86.23 | 0.58 | 10.9 | 7.8 | None |
| | 16b | | 27 | | | | Remission | 0 | 2 | 28 | 12.07 | 68.88 | 0.6 | 11.6 | NA | None |
| 17 | 17a | MPA | 53 | F | W | MPO | Active | 10 | 3 | 119.3 | 26.16 | 94.92 | 0.29 | 18 | 15.8 | MPD; |
| | | | | | | | | | | | | | | | | CYIV |
| | 17b | | 54 | | | | Remission | 0 | 2.2 | 10.5 | 112.28 | 338.63 | 0.44 | 10.4 | NA | CS; MPD; |
| | | | | | | | | | | | | | | | | CYIV; |
| | | | | | | | | | | | | | | | | RITUX |
| 18 | 18a | MPA | 57 | M | B | MPO | *Active | 20 | 3 | 140.1 | 252.54 | 366.86 | 0.25 | 15.4 | NA | CS; |
| | | | | | | | | | | | | | | | | RITUX; |
| | | | | | | | | | | | | | | | | PLEX |
| | 18b | | 57 | | | | Remission | 0 | NA | NA | 33.33 | 68.49 | 0.84 | NA | NA | CS |
| 19 | 19a | GPA | 59 | F | W | MPO | *Active | 3 | NA | NA | 740.67 | 2033.02 | 0.34 | 14 | 12.5 | CS |
| | 19b | | 60 | | | | Remission | 0 | 1.9 | 2.9 | 134.41 | 243.99 | 0.55 | 5.1 | NA | CS; CYIV |
| | 19c | | 61 | | | | Remission | 0 | NA | NA | 132.54 | 312.36 | NA | 9.1 | 5.3 | RITUX |
| 20 | 20a | Lim | 60 | F | B | MPO | Active | 6 | 1.7 | 3.4 | NA | NA | NA | 9.6 | 6.5 | RITUX |
| | 20b | | 60 | | | | Remission | 0 | 1.4 | 3.6 | 0.08 | 47.30 | 0.55 | 10.9 | 7.9 | RITUX |
| 21 | 21a | MPA | 69 | M | W | MPO | *Active | 12 | 1.8 | 108 | 103.07 | 342.52 | 0.64 | 9.6 | 7 | None |
| | 21b | | 69 | | | | Active | 16 | 0.7 | 64.9 | 19.91 | 86.63 | 0.85 | 3.8 | 2 | CS; |
| | | | | | | | | | | | | | | | | RITUX |
| | 21c | | 69 | | | | Remission | 0 | NA | NA | 183.38 | 626.70 | 0.58 | 10.4 | 7.9 | CS; |
| | | | | | | | | | | | | | | | | RITUX |
| 22 | 22a | GPA | 80 | M | W | MPO | Active | 6 | 2.8 | 45 | 12.83 | 150.77 | 0.6 | 10 | NA | None |
| | 22b | | 80 | | | | Remission | 0 | NA | NA | 41.43 | 411.60 | 0.71 | NA | NA | RITUX |
| 23 | 23a | GPA | 22 | F | W | MPO | Active | 3 | 1.7 | 9.4 | 31.68 | 115.42 | 0.6 | 5.2 | 3.4 | CS; MMF; |
| | | | | | | | | | | | | | | | | belumimab |
| | 23b | | 23 | | | | Remission | 0 | 1.3 | 11.6 | 22.34 | 41.05 | 0.34 | 6.9 | 5.6 | CYIV; |
| | | | | | | | | | | | | | | | | RITUX |
| 24 | 24a | GPA | 33 | F | U | MPO | Active | 12 | 4.3 | 57.9 | 10.25 | 74.86 | 0.36 | 6.6 | NA | CS |
| | 24b | | 34 | | | | Remission | 0 | 3.7 | 2.8 | 0.07 | 36.96 | 0.65 | 5.3 | 3.7 | None |
| 25 | 25a | EGPA | 51 | M | W | MPO | *Active | 24 | 2.7 | 85.6 | 2488.70 | 2171.77 | 0.51 | 18.1 | NA | CS |
| | 25b | | 52 | | | | Remission | 0 | 2.4 | 6.9 | 38.33 | 137.39 | 0.87 | 5.4 | 2.9 | AZA |
| 26 | 26a | GPA | 48 | F | U | MPO | Active | 6 | 2.4 | 15.9 | 1795.83 | 9560.15 | 0.47 | 11.7 | 9.6 | CS; CYIV |
| | 26b | | 48 | | | | Remission | 1 | 6.1 | 21.2 | 48.85 | 832.22 | 0.64 | 5.7 | 3.5 | CS; CYIV |
| 27 | 27a | GPA | 24 | F | W | MPO | *Active | 8 | 1.4 | 7 | 73.76 | 448.45 | 0.51 | 18.6 | 14.9 | CS |
| | 27b | | 25 | | | | Remission | 0 | 1.4 | 2.1 | 2.89 | 43.01 | 0.62 | 11.5 | 8.7 | CS |
| 28 | 28a | MPA | 72 | M | W | MPO | Active | 6 | 1.5 | 62.4 | 407.32 | 1199.96 | NA | 6.3 | 5.6 | CS; MMF |
| | 28b | | 73 | | | | Remission | 0 | NA | NA | 23.98 | 123.94 | 0.62 | NA | NA | MMF |
| 29 | 29a | MPA | 33 | M | W | MPO | Active | 8 | NA | NA | 55.13 | 317.00 | 0.57 | NA | NA | None |
| 30 | 30a | MPA | 80 | M | W | MPO | Active | 8 | NA | NA | 674.09 | 213.60 | 0.32 | NA | NA | CS; AZA |
| | 30b | | 81 | | | | Remission | 0 | 1.6 | 3.6 | 26.67 | 135.30 | 0.61 | 8 | 6 | CYIV |
| 31 | 31a | Lim | 83 | M | W | MPO | Active | 15 | 1.7 | 51.5 | 176.18 | 588.15 | 0.42 | 8.8 | NA | RITUX |
| 32 | 32a | MPA | 47 | M | H | MPO | Remission | 0 | 2.3 | 3.5 | 16.17 | 216.50 | 0.60 | 4.2 | 2.2 | AZA |
| | 32b | | 48 | | | | Remission | 0 | 4.6 | 18.7 | 13.81 | 123.64 | 0.48 | 7 | NA | None |
| | 32c | | 49 | | | | Remission | 0 | 2.6 | 17.5 | 23.58 | 290.17 | 0.65 | 8 | NA | None |
| 33 | 33a | Lim | 51 | M | W | MPO | Remission | 1 | NA | NA | 163.38 | 305.40 | 0.67 | 6.6 | 4.4 | AZA |
| | 33b | | 52 | | | | Remission | 0 | 1.5 | 35.7 | 133.98 | 187.97 | 0.75 | 7.8 | NA | None |
| | 33c | | 54 | | | | Remission | 0 | NA | NA | 99.93 | 238.48 | 0.92 | NA | NA | None |
| 34 | 34a | MPA | 21 | F | W | MPO | Active | 6 | 1 | 53 | NA | NA | NA | 6 | 3.2 | None |
| | 34b | | 25 | | | | Remission | 0 | 2.4 | 86.9 | 25.17 | 87.33 | 1.28 | 5.7 | 3.6 | MMF |
| | 34c | | 33 | | | | Remission | 0 | 2.1 | 51.5 | NA | NA | NA | 8.9 | NA | None |
| | 34d | | 38 | | | | Remission | 0 | NA | NA | 21.23 | 91.73 | 0.78 | NA | NA | None |
| 35 | 35a | Lim | 23 | F | W | MPO | Remission | 0 | 4.6 | 12.4 | 4.88 | 15.78 | 0.59 | 7.6 | NA | None |
| | 35b | | 25 | | | | Remission | 0 | 2.2 | 12.4 | 9.09 | 35.53 | 0.44 | NA | NA | None |
| 36 | 36a | Lim | 67 | M | W | MPO | Remission | 0 | 2.5 | 92.3 | 6.33 | 110.12 | NA | NA | NA | None |
| 37 | 37a | MPA | 42 | F | W | MPO | Remission | 0 | 2.2 | 10.8 | 166.44 | 421.52 | 0.5 | NA | NA | CS |
| 38 | 38a | MPA | 56 | F | W | MPO | Remission | 0 | 1.5 | 36 | 13.75 | 69.36 | 1.64 | 6.8 | 4.7 | None |
| 39 | 39a | GPA | 51 | F | W | MPO | Remission | 0 | 1.3 | 4.8 | NA | NA | NA | 6.1 | NA | None |
| 40 | 40a | Lim | 66 | F | B | MPO | Remission | 0 | NA | NA | NA | NA | NA | 6.2 | NA | None |
| 41 | 41a | MPA | 55 | F | A | PR3 | Active | 6 | 78.8 | 2.3 | NA | NA | NA | 6 | NA | MMF |
| | 41b | | 57 | | | | Remission | 0 | 21.4 | 1.3 | 5.28 | 39.88 | 0.73 | 4.6 | NA | MMF |
| | 41c | | 59 | | | | Active | 0 | 40 | 1 | 0.33 | 39.58 | 0.96 | 6.2 | 3.6 | MMF |
| 42 | 42a | GPA | 47 | M | W | PR3 | Active | 19 | 107.7 | 2.6 | 9.82 | 38.18 | 0.21 | 22.6 | 20.4 | CYIV; CS; |
| | | | | | | | | | | | | | | | | PLEX |
| | 42b | | 49 | | | | Remission | 0 | NA | NA | 30.34 | 120.19 | 0.54 | 9.3 | 6.4 | CS |
| | 42c | | 49 | | | | Remission | 0 | NA | NA | 18.11 | 67.88 | 0.73 | 8.2 | 5.6 | CS |
| 43 | 43a | MPA | 53 | M | W | PR3 | Remission | 0 | 145 | 1.6 | NA | NA | NA | 4.7 | 3.6 | CYIV; CS |
| | 43b | | 54 | | | | Active | 12 | 165 | 2.3 | NA | NA | NA | 6.4 | NA | AZA |
| 44 | 44a | MPA | 74 | F | W | PR3 | Active | 0 | 22.5 | 2.7 | NA | NA | NA | 2.3 | 1.8 | CYIV; CS |
| | 44b | | 79 | | | | Remission | 0 | 114.8 | 3.4 | 48.75 | 179.32 | 0.87 | 7.2 | 4.6 | AZA |
| | 44c | | 83 | | | | Remission | 0 | NA | NA | 20.43 | 32.43 | 1.35 | 6.3 | NA | None |
| 45 | 45a | GPA | 34 | M | B | PR3 | *Active | 13 | 136.2 | 3.1 | 34.91 | 61.88 | 0.48 | 19.5 | NA | MPD; |
| | | | | | | | | | | | | | | | | PLEX |
| | 45b | | 41 | | | | Remission | 0 | 40 | 3.5 | 9.97 | 26.05 | NA | 8.6 | NA | MMF; |
| | | | | | | | | | | | | | | | | CyA; CS |
| 46 | 46a | GPA | 50 | F | B | PR3 | *Active | 21 | 104.8 | 3.4 | 683.67 | 2008.83 | 0.33 | 16 | 14.6 | HD; MPD; |
| | | | | | | | | | | | | | | | | PLEX |
| | 46b | | 59 | | | | Remission | 0 | 29.3 | 5.7 | 4.23 | 23.75 | 0.47 | 6.6 | 5.1 | AZA |
| | 46c | | 60 | | | | Remission | 0 | NA | NA | NA | NA | NA | 7.2 | 4.9 | None |
| 47 | 47a | GPA | 70 | M | W | PR3 | Active | 0 | 123.7 | 3 | 32.81 | 62.24 | NA | 5.2 | NA | RITUX |
| | 47b | | 71 | | | | Remission | 0 | 85.5 | 1.5 | 43.15 | 39.83 | 1.19 | 7.8 | 3 | None |
| | 47c | | 71 | | | | Remission | 0 | NA | NA | 67.66 | 86.44 | 1.94 | 7.8 | NA | RITUX |
| 48 | 48a | MPA | 64 | M | W | PR3 | Active | 1 | 148.3 | 5 | 9.81 | 52.94 | 0.66 | 4.3 | 2.5 | AZA |
| | 48b | | 65 | | | | Remission | 0 | 67.8 | 2.4 | 12.27 | 70.84 | 0.71 | 4.9 | 3 | None |
| | 48c | | 66 | | | | Active | 1 | 161.7 | 3.5 | 0.08 | 45.86 | 0.69 | NA | NA | None |
| | 48d | | 67 | | | | Active | 7 | 147.9 | 1.9 | 0.16 | 7.19 | 0.5 | 7.2 | NA | CS; AZA |
| | 48e | | 67 | | | | Remission | 0 | 36.7 | 2.6 | 20.77 | 184.16 | 0.81 | 4.8 | NA | CS; |
| | | | | | | | | | | | | | | | | RITUX |
| 49 | 49a | MPA | 71 | F | W | PR3 | Active | 2 | NA | NA | 55.84 | 899.21 | NA | 8.8 | NA | CS; MMF |
| | 49b | | 72 | | | | Remission | 0 | NA | NA | 67.62 | 445.37 | 0.76 | 9.7 | 6.1 | CS; MMF |
| 50 | 50a | GPA | 32 | F | W | PR3 | Remission | 0 | 50.3 | 4 | 42.44 | 62.88 | 0.87 | 3.6 | 2.3 | AZA |
| | 50b | | 33 | | | | Active | 6 | 69.9 | 5 | 17.08 | 22.33 | 1.01 | NA | NA | AZA |
| 51 | 51a | GPA | 19 | M | W | PR3 | Remission | 0 | NA | NA | 685.64 | 297.81 | 0.40 | NA | NA | CS; AZA; |
| | | | | | | | | | | | | | | | | CyA |
| | 51b | | 20 | | | | Active | 6 | 2.3 | 1.7 | 1849.11 | 2760.90 | 0.40 | 13.5 | 11.8 | CS; AZA |
| 52 | 52a | MPA | 53 | F | W | PR3 | Active | 3 | 108.1 | 2.4 | 4.88 | 27.08 | 0.78 | 7.6 | 5.1 | MMF |
| | 52b | | 54 | | | | Remission | 0 | 50.6 | 2.4 | 5.71 | 81.44 | 0.42 | 6.5 | 4.6 | MMF; |
| | | | | | | | | | | | | | | | | RITUX |
| 53 | 53a | GPA | 57 | F | W | PR3 | Active | 9 | 102.4 | 3.2 | 76.19 | 481.00 | 0.29 | 9.7 | 8.9 | CS |
| | 53b | | 58 | | | | Remission | 0 | 111.4 | 1.4 | 17.16 | 63.65 | 1.57 | 6.6 | 4.2 | None |
| 54 | 54a | GPA | 50 | M | W | PR3 | Active | 3 | 16.1 | 2.5 | 26.03 | 196.25 | 0.43 | 9.2 | 8.1 | CS; CYIV; |
| | | | | | | | | | | | | | | | | RITUX; |
| | | | | | | | | | | | | | | | | MMF |
| | 54b | | 52 | | | | Remission | 0 | 12.8 | 1.1 | 11.28 | 29.81 | 0.78 | 6.5 | 4.6 | RITUX; |
| | | | | | | | | | | | | | | | | AZA |
| | 54c | | 53 | | | | Active | 4 | 11.5 | 0.9 | 5.42 | 75.76 | 0.58 | 6.3 | 4.7 | None |
| | 54d | | 54 | | | | Remission | 0 | 5 | 1.5 | 6.61 | 56.19 | 0.72 | NA | NA | RITUX |
| 55 | 55a | MPA | 69 | M | W | PR3 | Remission | 0 | 83.7 | 2.7 | 13.63 | 78.93 | 0.82 | 6.1 | 4.5 | MMF |
| | 55b | | 72 | | | | Active | 7 | 124.6 | 2.3 | 30.13 | 373.99 | 0.6 | 5.7 | 3.7 | CS; MMF |
| | 55c | | 73 | | | | Remission | 0 | NA | NA | 55.99 | 175.44 | 0.44 | NA | NA | RITUX |
| 56 | 56a | GPA | 56 | M | W | PR3 | Remission | 0 | 20.4 | 2.2 | 11.81 | 69.28 | NA | 4.4 | 2.5 | RITUX; |
| | | | | | | | | | | | | | | | | MMF |
| | 56b | | 57 | | | | Active | 6 | 59.6 | 2 | 6.20 | 61.76 | 0.58 | 5.2 | 3 | MMF |
| | 56c | | 59 | | | | Active | 6 | 84.1 | 1.1 | 5.43 | 60.07 | 0.72 | NA | NA | MMF |
| | 56d | | 59 | | | | Remission | 0 | 27.8 | 1.9 | 4.92 | 52.04 | 1.06 | 5.6 | 3.5 | RITUX |
| 57 | 57a | MPA | 58 | F | W | PR3 | *Active | 8 | 157.3 | 1.9 | 7.47 | 131.35 | 0.55 | 13.6 | NA | CS |
| | 57b | | 60 | | | | Remission | 0 | 28 | 1.5 | 0.36 | 77.42 | 0.71 | 5 | 2.8 | RITUX |
| 58 | 58a | MPA | 65 | M | W | PR3 | *Active | 18 | 149.9 | 1.4 | 329.28 | 471.68 | 0.43 | 1.4 | NA | MPD; |
| | | | | | | | | | | | | | | | | PLEX |
| | 58b | | 66 | | | | Remission | 0 | 26 | 1.2 | 14.97 | 165.41 | 0.73 | 8.6 | 5.9 | RITUX |
| | 58c | | 67 | | | | Active | 1 | 27.7 | 1.7 | 82.16 | 193.62 | 0.46 | 8.6 | 5.6 | None |
| | 58d | | 67 | | | | Remission | 0 | 7.6 | 1.9 | 16.96 | 83.91 | 0.67 | 6.8 | 4.3 | RITUX |
| 59 | 59a | GPA | 36 | M | W | PR3 | Active | 5 | 92.1 | 2.4 | 568.03 | 2001.14 | 0.44 | 8.8 | 7.8 | MPD |
| | 59b | | 38 | | | | Remission | 0 | 33 | 1.6 | 27.39 | 102.28 | 0.62 | 7 | 4.7 | None |
| | 59c | | 39 | | | | Remission | 0 | 15.5 | 1.4 | 9.02 | 92.43 | 0.61 | 11.2 | 8.9 | None |
| 60 | 60a | MPA | 29 | F | W | PR3 | Active | 12 | 121.4 | 1.5 | 35.65 | 79.95 | 0.67 | 10.8 | NA | CYIV |
| | 60b | | 29 | | | | Remission | 0 | NA | NA | 8.38 | 34.27 | 0.52 | 6.1 | 4.5 | AZA |
| | 60c | | 30 | | | | Remission | 0 | 104.5 | 1.9 | 4.43 | 53.07 | 0.91 | 3.8 | NA | AZA |
| 61 | 61a | MPA | 45 | F | B | PR3 | Active | 4 | NA | NA | 14.53 | 176.10 | NA | NA | NA | CS; MTX |
| | 61b | | 45 | | | | Active | 0 | 92.9 | 2.1 | 4.43 | 33.32 | 1.04 | 9.6 | 7.5 | CS; |
| | | | | | | | | | | | | | | | | RITUX |
| | 61c | | 47 | | | | Remission | 0 | 109.9 | 1.9 | 2.49 | 40.21 | 0.48 | 7.6 | 6 | RITUX |
| 62 | 62a | GPA | 37 | M | W | PR3 | Active | 0 | 70.2 | 2.2 | 30.66 | 92.71 | 0.57 | NA | NA | None |
| | 62b | | 38 | | | | Remission | 0 | 47.6 | 1.5 | 13.34 | 154.49 | 0.71 | 8.3 | 5.7 | None |
| 63 | 63a | GPA | 73 | F | W | PR3 | Active | 0 | 3.3 | 1.5 | 90.61 | 203.09 | 0.69 | 5.8 | NA | CS; CYPO |
| | 63b | | 74 | | | | Remission | 0 | 2.6 | 4.6 | 6.91 | 43.46 | 1.22 | 6.7 | 4.7 | AZA |
| 64 | 64a | GPA | 51 | M | W | PR3 | *Active | 7 | NA | NA | 2207.36 | 5354.39 | 0.45 | 26.6 | NA | CS |
| | 64b | | 52 | | | | Remission | 0 | NA | NA | NA | NA | NA | 11.3 | 6.9 | CS; |
| | | | | | | | | | | | | | | | | RITUX |
| 65 | 65a | GPA | 76 | M | W | PR3 | Active | 22 | 200 | 1.6 | 1185.90 | 2459.09 | 0.32 | 11.4 | NA | MPD; |
| | | | | | | | | | | | | | | | | PLEX |
| | 65b | | 76 | | | | Remission | 0 | 114.7 | 1.1 | 1494.68 | 2642.21 | 0.43 | 10.7 | 9.6 | CS; CYIV; |
| | | | | | | | | | | | | | | | | RITUX |
| | 65c | | 77 | | | | Remission | 0 | 124.1 | 1.4 | 52.12 | 51.60 | 0.54 | 5.9 | 3.4 | None |
| 66 | 66a | GPA | 82 | F | W | PR3 | Active | 19 | 130 | 1.4 | NA | NA | NA | 9.2 | 8.3 | CS; MMF |
| | 66b | | 82 | | | | Remission | 0 | 87.4 | 1.3 | 0.20 | 10.71 | 0.68 | 5.2 | 3.6 | CS; |
| | | | | | | | | | | | | | | | | RITUX |
| 67 | 67a | GPA | 60 | M | W | PR3 | *Active | 13 | 79.1 | 1.4 | 44.78 | 108.07 | 0.39 | 7.3 | NA | CYPO |
| | 67b | | 60 | | | | Remission | 0 | NA | NA | 152.85 | 593.24 | 0.4 | 9.4 | 8 | CS; |
| | | | | | | | | | | | | | | | | RITUX |
| 68 | 68a | MPA | 69 | M | W | PR3 | Active | 17 | 49 | 1.7 | 534.11 | 1705.02 | 0.44 | 11.4 | 9 | CS; CYPO |
| | 68b | | 71 | | | | Remission | 0 | 22 | 2.6 | 9.03 | 87.86 | 1.03 | 7.5 | NA | None |
| 69 | 69a | GPA | 55 | M | W | PR3 | Active | 4 | 116.9 | 5.9 | 14.85 | 28.68 | 0.18 | 15 | 13.9 | CS; |
| | | | | | | | | | | | | | | | | RITUX |
| | 69b | | 58 | | | | Remission | 0 | 128.9 | 5.3 | 3.39 | 22.28 | 0.85 | 8.4 | 4.9 | None |
| 70 | 70a | GPA | 56 | F | W | PR3 | *Active | 21 | 99 | 1.8 | 1615.41 | 2695.97 | 0.28 | 21.8 | 19.5 | CS |
| | 70b | | 58 | | | | Remission | 0 | 126.8 | 1.7 | 108.51 | 158.83 | 0.56 | 7.4 | 5.2 | None |
| 71 | 71a | MPA | 79 | M | O | PR3 | *Active | 16 | 153.4 | 5.7 | 19.85 | 41.30 | 0.16 | 5.7 | NA | MPD |
| | 71b | | 81 | | | | Remission | 0 | 100.8 | 3.8 | 3.31 | 14.24 | 0.38 | 2.9 | 2.3 | CS; AZA |
| 72 | 72a | GPA | 70 | F | W | PR3 | *Active | 17 | 145 | 1.7 | 27.77 | 80.64 | 0.14 | 14.9 | NA | CS; CYIV |
| | 72b | | 70 | | | | Remission | 0 | 99 | 0.8 | 240.05 | 739.77 | 0.33 | 12.5 | 11.4 | CS; |
| | | | | | | | | | | | | | | | | RITUX |
| 73 | 73a | GPA | 46 | M | W | PR3 | Remission | 0 | NA | NA | 46.90 | 106.89 | 0.3 | 10.8 | 9.3 | CS; |
| | | | | | | | | | | | | | | | | RITUX |
| 74 | 74a | GPA | 61 | M | W | PR3 | Active | 14 | 42.5 | 1.5 | 46.47 | 321.69 | 0.68 | 6.8 | 4.7 | CS |
| 75 | 75a | GPA | 50 | F | W | PR3 | Active | 2 | 4.1 | 1.2 | 9.65 | 83.23 | 0.68 | 6.4 | 4.7 | MMF |
| 76 | 76a | GPA | 32 | M | W | PR3 | *Active | 7 | 143.4 | 6.4 | 54.97 | 68.96 | 0.75 | NA | NA | None |
| 77 | 77a | MPA | 33 | F | W | PR3 | Remission | 0 | 95.3 | 2.1 | 17.07 | 114.31 | 1.76 | 8.4 | NA | None |
| | 77b | | 34 | | | | Remission | 0 | 33.2 | 1.6 | 118.40 | 494.86 | 0.7 | 11 | 7.3 | None |
| 78 | 78a | MPA | 24 | F | W | PR3 | Remission | 0 | 1.2 | 2.9 | NA | NA | NA | NA | NA | CYPO |
| | 78b | | 30 | | | | Remission | 0 | 2.8 | 1.1 | 4.84 | 40.92 | 0.58 | 7.3 | NA | None |
| 79 | 79a | GPA | 71 | M | W | PR3 | Remission | 0 | 1.2 | 2.9 | NA | NA | 0.91 | 8.4 | 5.1 | None |
| | 79b | | 74 | | | | Remission | 0 | 2.7 | 3.1 | 34.68 | 162.00 | 0.71 | NA | NA | None |
| | 79c | | 77 | | | | Remission | 0 | 14.6 | 1.8 | 16.30 | 122.37 | 0.5 | NA | NA | None |
| 80 | 80a | GPA | 76 | M | B | PR3 | Remission | 0 | NA | NA | 135.61 | 234.91 | 1 | 6.3 | 4.5 | None |
| | 80b | | 77 | | | | Remission | 0 | 3.1 | 1.9 | 77.39 | 59.55 | 0.72 | 9.1 | 6.2 | None |
| 81 | 81a | GPA | 67 | M | W | PR3 | Remission | 0 | 36.3 | 1.7 | 14.32 | 77.42 | 0.87 | 7.2 | NA | None |
| 82 | 82a | GPA | 40 | M | O | PR3 | Remission | 0 | 200 | 1.6 | 16.61 | 240.41 | 0.98 | 10 | 4.8 | None |

PR3, proteinase 3; MPO, myeloperoxidase; MPA, microscopic polyangiitis; GPA, granulomatosis with polyangiitis; EGPA, eosinophilic granulomatosis with polyangiitis; Lim, renal limited vasculitis; NA, not available; W, white; B, African-American; U, unknown; H, Hispanic; A, Asian; O, other; BVAS; Birmingham Vasculitis Activity Score; WBC, white blood cells; PMN, polymorphonuclear cells.
Therapy: MMF, mycophenolate mofetil; CyA, cyclosporine A; HD, hemodialysis; AZA, azathioprine; PLEX, plasmapheresis; CS, corticosteroids; CYIV, intravenous cyclophosphamide; CYPO, oral cyclophosphamide; MPD, methylprednisolone; MTX, methotrexate; RITUX, rituximab.

TABLE 6
Primers used for Taqman mRNA expression studies and DNA
methylation studies

| Gene | Primer |
|---|---|
| PRTN3 | Forward 5′-TGT CAC CGT GGT CAC CTT CTT-3′ |
| (Taqman) | (SEQ ID NO: 1) |
| | Reverse 5′-CCC CAG ATC ACG AAG GAG TCT AT-3′ |
| | (SEQ ID NO: 2) |
| | Probe FAM-TTG CAC TTT CGT CCC TCG CCG-TAMRA |
| | (SEQ ID NO: 3) |
| MPO | Forward 5′-CCA GGA AGC CCG GAA GAT-3' |
| (Taqman) | (SEQ ID NO: 4) |
| | Reverse 5′-CGG AAG GCA TTG GTG AAG A-3′ |
| | (SEQ ID NO: 5) |
| | Probe FAM-TGC CCA CGT ACC GTT CCT ACA ATG ACT C-TAMRA |
| | (SEQ ID NO: 6) |
| DNMT1 | Thermo Fisher cat. #4331182 |
| (Taqman) | |
| PRTN3 | Forward 5′-aggaagagagTTAAAGGGGGAAGAAAATTTTTAGA-3′ |
| (amplicon 5, | (SEQ ID NO: 7) |
| methy1ation) | Reverse 5′-cagtaatacgactcactatagggagaaggctATTACCC |
| | AATACCCAAACTAAATCC-3′ (SEQ ID NO: 8) |
| PRTN3 | Forward 5′-aggaagagagAAGTGTTGTTGGGTGGGTTTTT-3′ |
| (amplicon 20, | (SEQ ID NO: 9) |
| methylation) | Reverse 5′-cagtaatacgactcactatagggagaaggct |
| | AACACCTTAATCCACCCCAACTT-3′ (SEQ ID NO: 10) |
| PRTN3 | Forward 5′-aggaagagagTTGTGATGGTATTATTTAAGGAATAGATTT-3′ |
| (amplicon 22, | (SEQ ID NO: 11) |
| methylation) | Reverse 5′-cagtaatacgactcactatagggagaaggct |
| | CCTCCCCTCCCTATATAAAAAAAAC-3′ (SEQ ID NO: 12) |
| MPO | Forward 5′-aggaagagagGTTGGGGGTGGTTGTAGGAAT-3′ |
| (amplicon 11, | (SEQ ID NO: 13) |
| methylation) | Reverse 5′-cagtaatacgactcactatagggagaaggct |
| | CAACTAACCCCATTACATAAACATAAA-3′ (SEQ ID NO: 14) |
| MPO | Forward 5′-aggaagagagAAGTTGATTTGTTAGGAAGTAGGGG-3′ |
| (Amplicon 12, | (SEQ ID NO: 15) |
| methylation) | Reverse 5′-cagtaatacgactcactatagggagaaggct |
| | AACCTCTCTCTATACCTCAAATCCC-3′ (SEQ ID NO: 16) |
| LTF | Forward 5′-GGA GTT TTG TTT TGT TTT AGG GTT T-3′ |
| (amplicon 3, | (SEQ ID NO: 17) |
| methylation) | Reverse 5′-CTC CTA TTC CTC CCC ATA TAA AAA A-3′ |
| | (SEQ ID NO: 18) |
| ELANE | Forward 5′-TGG TGG GGG ATT TAG AGG TT-3′ |
| (amplicon 5, | (SEQ ID NO: 19) |
| methylation | Reverse 5′-AAC TAC AAA AAC ACC ATA AAA AAC CA-3′ |
| | (SEQ ID NO: 20) |

TABLE 7
Gene coordinates for each of the seven loci studied

| Gene_Amplicon | Chromosome | Browser position |
|---|---|---|
| PRTN3_005 | 19 | 840498 840945 |
| PRTN3_022 | 19 | 847822 848215 |
| PRTN3_020 | 19 | 843571 843964 |
| MPO_11 | 17 | 56356480 56356874 |
| MPO_12 | 17 | 56355180 56355526 |
| LTF_003 | 3 | 46506126 46506547 |
| ELA_005 | 19 | 852792 852954 |

That which is claimed is:

1. A method of identifying a subject having anti-neutrophilic cytoplasmic autoantibody (ANCA)-associated vasculitis (AAV) as having an increased likelihood of relapse of (AAV), comprising:
a) determining the methylation status of CpG dinucleotides in the promoter of a PRTN3 gene in a first sample obtained from the subject at a first time point;
b) determining the methylation status of CpG dinucleotides in the promoter of a PRTN3 gene in a subsequent sample obtained from the subject at a subsequent time point following the first time point; and
c) comparing the methylation status of (a) with the methylation status of (b), wherein a decrease in the methylation status of (b) relative to (a) identifies the subject as having an increased likelihood of relapse of AAV.

2. The method of claim 1, further comprising initiating and/or enhancing a treatment regimen of the subject.

3. The method of claim 1, wherein the treatment regimen is immunosuppression, DNA methylation, reduction of or elimination of or avoidance of therapy that decreases DNA methylation, and any combination thereof.

4. A method of guiding treatment of a subject having AAV, comprising
a) determining the methylation status of CpG dinucleotides in the promoter of a PRTN3 gene in a first sample obtained from the subject at a first time point;
b) determining the methylation status of CpG dinucleotides in the promoter of a PRTN3 gene in a subsequent sample obtained from the subject at a subsequent time point following the first time point;
c) comparing the methylation status of (a) with the methylation status of (b), wherein a decrease in the methylation status of (b) relative to (a) identifies the subject as having an increased likelihood of relapse of AAV and an increase or no change in methylation status of (b) relative to (a) identifies the subject as not having an increased risk of relapse of AAV; and
d) initiating and/or enhancing a treatment regimen in a subject identified as having an increased likelihood of relapse of AAV, or not initiating or enhancing a treatment regimen and/or reducing a treatment regimen in a subject identified as not having an increased risk of relapse of AAV.

5. A method of evaluating response to treatment for AAV in a subject having AAV, comprising:
a) determining the methylation status of CpG dinucleotides in the promoter of a PRTN3 gene in a first sample obtained from the subject at a first time point prior to administration of a treatment for AAV;
b) administering to the subject the treatment for AAV;
c) determining the methylation status of CpG dinucleotides in the promoter of a PRTN3 gene in a subsequent sample obtained from the subject at a subsequent time point following the treatment for AAV; and
d) comparing the methylation status of (a) with the methylation status of (b), wherein an increase in the methylation status of (b) relative to (a) identifies the subject as having a positive response to treatment and a decrease or no change in the methylation status of (b) relative to (a) identifies the subject as having no response to treatment or a negative response to treatment.

6. The method of claim 5, wherein the treatment for AAV is administration of an agent that increases DNA methylation, reduction of, elimination of and/or avoidance of treatment that decreases DNA methylation, an immunosuppressive agent, and any combination thereof.

7. A method of reducing the likelihood of relapse in a subject in remission from AAV, comprising:
a) determining the methylation status of CpG dinucleotides in the promoter of a PRTN3 gene in a first sample obtained from the subject at a first time point;
b) determining the methylation status of CpG dinucleotides in the promoter of a PRTN3 gene in a subsequent sample obtained from the subject at a subsequent time point;
c) comparing the methylation status of (a) with the methylation status of (b), wherein a decrease in the methylation status of (b) relative to (a) identifies the subject as having an increased risk of relapse of AAV; and
d) initiating and/or enhancing a treatment regimen of the subject.

8. The method of claim 7, wherein the treatment regimen is immunosuppression, DNA methylation, reduction of or elimination of or avoidance of therapy that decreases DNA methylation, and any combination thereof.

9. A kit of reagents for the detection of methylation of one or more CpG dinucleotides in the promoter of a PRTN3 gene.
